SEQUENCE LISTING (11 sequences)

SEQ ID NO: 1 - DNA, 25 bp, Artificial Sequence
  Description of Artificial Sequence Synthetic untranslated oligonucleotide
gttcttaaggcacaggaactgggac

SEQ ID NO: 2 - DNA, 3222 bp, Homo sapiens
atgaagacgttgctgttggacttggctttgtggtcactgctcttccagcccgggtggctgtcctttagttcccaggtgagtcagaactgccacaatggcagctatgaaatcagcgtcctgatgatgggcaactcagcctttgcagagcccctgaaaaacttggaagatgcggtgaatgaggggctggaaatagtgagaggacgtctgcaaaatgctggcctaaatgtgactgtgaacgctactttcatgtattcggatggtctgattcataactcaggcgactgccggagtagcacctgtgaaggcctcgacctactcaggaaaatttcaaatgcacaacggatgggctgtgtcctcatagggccctcatgtacatactccaccttccagatgtaccttgacacagaattgagctaccccatgatctcagctggaagttttggattgtcatgtgactataaagaaaccttaaccaggctgatgtctccagctagaaagttgatgtacttcttggttaacttttggaaaaccaacgatctgcccttcaaaacttattcctggagcacttcgtatgtttacaagaatggtacagaaactgaggactgtttctggtaccttaatgctctggaggctagcgtttcctatttctcccacgaactcggctttaaggtggtgttaagacaagataaggagtttcaggatatcttaatggaccacaacaggaaaagcaatgtgattattatgtgtggtggtccagagttcctctacaagctgaagggtgaccgagcagtggctgaagacattgtcattattctagtggatcttttcaatgaccagtacttggaggacaatgtcacagcccctgactatatgaaaaatgtccttgttctgacgctgtctcctgggaattcccttctaaatagctctttctccaggaatctatcaccaacaaaacgagactttgctcttgcctatttgaatggaatcctgctctttggacatatgctgaagatatttcttgaaaatggagaaaatattaccacccccaaatttgctcatgctttcaggaatctcacttttgaagggtatgacggtccagtgaccttggatgactggggggatgttgacagtaccatggtgcttctgtatacctctgtggacaccaagaaatacaaggttcttttgacctatgatacccacgtaaataagacctatcctgtggatatgagccccacattcacttggaagaactctaaacttcctaatgatattacaggccggggccctcagatcctgatgattgcagtcttcaccctcactggagctgtggtgctgctcctgctcgtcgctctcctgatgctcagaaaatatagaaaagattatgaacttcgtcagaaaaaatggtcccacattcctcctgaaaatatctttcctctggagaccaatgagaccaatcatgttagcctcaagatcgatgatgacaaaagacgagatacaatccagagactacgacagtgcaaatacgacaaaaagcgagtgattctcaaagatctcaagcacaatgatggtaatttcactgaaaaacagaagatagaattgaacaagttgcttcagattgactattacaacctgaccaagttctacggcacagtgaaacttgataccatgatcttcggggtgatagaatactgtgagagaggatccctccgggaagttttaaatgacacaatttcctaccctgatggcacattcatggattgggagtttaagatctctgtcttgtatgacattgctaagggaatgtcatatctgcactccagtaagacagaagtccatggtcgtctgaaatctaccaactgcgtagtggacagtagaatggtggtgaagatcactgattttggctgcaattccattttacctccaaaaaaggacctgtggacagctccagagcacctccgccaagccaacatctctcagaaaggagatgtgtacagctatgggatcatcgcacaggagatcattctgcggaaagaaaccttctacactttgagctgtcgggaccggaatgagaagattttcagagtggaaaattccaatggaatgaaacccttccgcccagatttattcttggaaacagcagaggaaaaagagctagaagtgtacctacttgtaaaaaactgttgggaggaagatccagaaaagagaccagatttcaaaaaaattgagactacacttgccaagatatttggactttttcatgaccaaaaaaatgaaagctatatggataccttgatccgacgtctacagctatattctcgaaacctggaacatctggtagaggaaaggacacagctgtacaaggcagagagggacagggctgacagacttaactttatgttgcttccaaggctagtggtaaagtctctgaaggagaaaggctttgtggagccggaactatatgaggaagttacaatctacttcagtgacattgtaggtttcactactatctgcaaatacagcacccccatggaagtggtggacatgcttaatgacatctataagagttttgaccacattgttgatcatcatgatgtctacaaggtggaaaccatcggtgatgcgtacatggtggctagtggtttgcctaagagaaatggcaatcggcatgcaatagacattgccaagatggccttggaaatcctcagcttcatggggacctttgagctggagcatcttcctggcctcccaatatggattcgcattggagttcactctggtccctgtgctgctggagttgtgggaatcaagatgcctcgttattgtctatttggagatacggtcaacacagcctctaggatggaatccactggcctccctttgagaattcacgtgagtggctccaccatagccatcctgaagagaactgagtgccagttcctttatgaagtgagaggagaaacatacttaaagggaagaggaaatgagactacctactggctgactgggatgaaggaccagaaattcaacctgccaacccctcctactgtggagaatcaacagcgtttgcaagcagaattttcagacatgattgccaactctttacagaaaagacaggcagcagggataagaagccaaaaacccagacgggtagccagctataaaaaaggcactctggaatacttgcagctgaataccacagacaaggagagcacctatttttaa

SEQ ID NO: 3 - protein, 1073 aa, Homo sapiens
MKTLLLDLALWSLLFQPGWLSFSSQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYLEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQILMIAVFTLTGAVVLLLLVALLMLRKYRKDYELRQKKWSHIPPENIFPLETNETNHVSLKIDDDKRRDTIQRLRQCKYDKKRVILKDLKHNDGNFTEKQKIELNKLLQIDYYNLTKFYGTVKLDTMIFGVIEYCERGSLREVLNDTISYPDGTFMDWEFKISVLYDIAKGMSYLHSSKTEVHGRLKSTNCVVDSRMVVKITDFGCNSILPPKKDLWTAPEHLRQANISQKGDVYSYGIIAQEIILRKETFYTLSCRDRNEKIFRVENSNGMKPFRPDLFLETAEEKELEVYLLVKNCWEEDPEKRPDFKKIETTLAKIFGLFHDQKNESYMDTLIRRLQLYSRNLEHLVEERTQLYKAERDRADRLNFMLLPRLVVKSLKEKGFVEPELYEEVTIYFSDIVGFTTICKYSTPMEVVDMLNDIYKSFDHIVDHHDVYKVETIGDAYMVASGLPKRNGNRHAIDIAKMALEILSFMGTFELEHLPGLPIWIRIGVHSGPCAAGVVGIKMPRYCLFGDTVNTASRMESTGLPLRIHVSGSTIAILKRTECQFLYEVRGETYLKGRGNETTYWLTGMKDQKFNLPTPPTVENQQRLQAEFSDMIANSLQKRQAAGIRSQKPRRVASYKKGTLEYLQLNTTDKESTYF

SEQ ID NO: 4 - DNA, 3186 bp, Homo sapiens
atgccggggccccggcgccccgctggctcccgcctgcgcctgctcctgctcctgctgctgccgccgctgctgctgctgctccggggcagccacgcgggcaacctgacggtagccgtggtactgccgctggccaatacctcgtacccctggtcgtgggcgcgcgtgggacccgccgtggagctggccctggcccaggtgaaggcgcgccccgacttgctgccgggctggacggtccgcacggtgctgggcagcagcgaaaacgcgctgggcgtctgctccgacaccgcagcgcccctggccgcggtggacctcaagtgggagcacaaccccgctgtgttcctgggccccggctgcgtgtacgccgccgccccagtggggcgcttcaccgcgcactggcgggtcccgctgctgaccgccggcgccccggcgctgggcttcggtgtcaaggacgagtatgcgctgaccacccgcgcggggcccagctacgccaagctgggggacttcgtggcggcgctgcaccgacggctgggctgggagcgccaagcgctcatgctctacgcctaccggccgggtgacgaagagcactgcttcttcctcgtggaggggctgttcatgcgggtccgcgaccgcctcaatattacggtggaccacctggagttcgccgaggacgacctcagccactacaccaggctgctgcggaccatgccgcgcaaaggccgagttatctacatctgcagctcccctgatgccttcagaaccctcatgctcctggccctggaagctggcttgtgtggggaggactacgttttcttccacctggatatctttgggcaaagcctgcaaggtggacagggccctgctccccgcaggccctgggagagaggggatgggcaggatgtcagtgcccgccaggcctttcaggctgccaaaatcattacatataaagacccagataatcccgagtacttggaattcctgaagcagttaaaacacctggcctatgagcagttcaacttcaccatggaggatggcctggtgaacaccatcccagcatccttccacgacgggctcctgctctatatccaggcagtgacggagactctggcacatgggggaactgttactgatggggagaacatcactcagcggatgtggaaccgaagctttcaaggtgtgacaggatacctgaaaattgatagcagtggcgatcgggaaacagacttctccctctgggatatggatcccgagaatggtgccttcagggttgtactgaactacaatgggacttcccaagagctggtggctgtgtcggggcgcaaactgaactggcccctggggtaccctcctcctgacatccccaaatgtggctttgacaacgaagacccagcatgcaaccaagatcacctttccaccctggaggtgctggctttggtgggcagcctctccttgctcggcattctgattgtctccttcttcatatacaggaagatgcagctggagaaggaactggcctcggagctgtggcgggtgcgctgggaggacgttgagcccagtagccttgagaggcacctgcggagtgcaggcagccggctgaccctgagcgggagaggctccaattacggctccctgctaaccacagagggccagttccaagtctttgccaagacagcatattataagggcaacctcgtggctgtgaaacgtgtgaaccgtaaacgcattgagctgacacgaaaagtcctgtttgaactgaagcatatgcgggatgtgcagaatgaacacctgaccaggtttgtgggagcctgcaccgacccccccaatatctgcatcctcacagagtactgtccccgtgggagcctgcaggacattctggagaatgagagcatcaccctggactggatgttccggtactcactcaccaatgacatcgtcaagggcatgctgtttctacacaatggggctatctgttcccatgggaacctcaagtcatccaactgcgtggtagatgggcgctttgtgctcaagatcaccgactatgggctggagagcttcagggacctggacccagagcaaggacacaccgtttatgccaaaaagctgtggacggcccctgagctcctgcgaatggcttcaccccctgtgcggggctcccaggctggtgacgtatacagctttgggatcatccttcaggagattgccctgaggagtggggtcttccacgtggaaggtttggacctgagccccaaagagatcatcgagcgggtgactcggggtgagcagccccccttccggccctccctggccctgcagagtcacctggaggagttggggctgctcatgcagcggtgctgggctgaggacccacaggagaggccaccattccagcagatccgcctgacgttgcgcaaatttaacagggagaacagcagcaacatcctggacaacctgctgtcccgcatggagcagtacgcgaacaatctggaggaactggtggaggagcggacccaggcatacctggaggagaagcgcaaggctgaggccctgctctaccagatcctgcctcactcagtggctgagcagctgaagcgtggggagacggtgcaggccgaagcctttgacagtgttaccatctacttcagtgacattgtgggtttcacagcgctgtcggcggagagcacacccatgcaggtggtgaccctgctcaatgacctgtacacttgctttgatgctgtcatagacaactttgatgtgtacaaggtggagacaattggcgatgcctacatggtggtgtcagggctccctgtgcggaacgggcggctacacgcctgcgaggtagcccgcatggccctggcactgctggatgctgtgcgctccttccgaatccgccaccggccccaggagcagctgcgcttgcgcattggcatccacacaggacctgtgtgtgctggagtggtgggactgaagatgccccgttactgtctctttggggatacagtcaacacagcctcaagaatggagtctaatggggaagccctgaagatccacttgtcttctgagaccaaggctgtcctggaggagtttggtggtttcgagctggagcttcgaggggatgtagaaatgaagggcaaaggcaaggttcggacctactggctccttggggagagggggagtagcacccgaggctga

SEQ ID NO: 5 - DNA, 3144 bp, Homo sapiens
atggcgctgccatcacttctgctgttggtggcagccctggcaggtggggtgcgtcctcccggggcgcggaacctgacgctggcggtggtgctgccagaacacaacctgagctatgcctgggcctggccacgggtgggacccgctgtggcactagctgtggaggctctgggccgggcactgcccgtggacctgcggtttgtcagctccgaactggaaggcgcctgctctgagtacctggcaccgctgagcgctgtggacctcaagctgtaccatgaccccgacctgctgttaggtcccggttgcgtgtaccctgctgcctctgtggcccgctttgcctcccactggcgccttcccctgctgactgcgggtgctgtggcctctggtttttcggctaagaatgaccattatcgtaccctggttcgcactggcccctctgctcccaagctgggtgagtttgtggtgacactacacgggcacttcaattggactgcccgtgctgccttgctgtacctggatgctcgcacagatgaccggcctcactacttcaccatcgagggcgtctttgaggccctgcagggcagcaacctcagtgtgcagcaccaggtgtatgcccgagagccagggggccccgagcaggccacccacttcatccgggccaacgggcgcattgtgtatatctgcggccctctggagatgctgcatgagatcctgcttcaggcccagagggagaatctgaccaatggggattatgtcttcttttacctggatgtctttggggagagtctccgtgcaggccccacacgtgctacaggccggccctggcaggacaatcgcacccgggaacaggcccaggccctcagagaggcctttcagactgtattggtgatcacgtaccgagaacccccaaatcctgagtatcaggaattccagaatcgtctgctgataagagcccgggaagactttggtgtggagctgggcccttccctgatgaacctcatcgctggctgcttctatgatgggatcctgctatatgctgaagtcctgaatgagacaatacaggaaggaggcacccgggaggatggacttcgaattgtggaaaagatgcagggacgaagatatcacggtgtaactgggctggttgtcatggacaagaacaatgaccgagagactgactttgtcctctgggccatgggagacctggattctggggactttcagcctgcagcccactactcgggagctgagaagcagatttggtggacgggacggcctattccctgggtgaagggggctcctccctcggacaatcccccctgtgcctttgacttggacgacccatcctgtgataaaactccactttcaaccctggcaattgtggctctgggcacaggaatcaccttcatcatgtttggtgtttccagcttcctaattttccgaaagctgatgctggagaaggagctggctagcatgttgtggcgtattcgctgggaagaactgcagtttggcaactcagagcgttatcacaaaggtgcaggcagtcgcctcacactgtcgctgcggggatccagttacggctcgctcatgacagcccatgggaaataccagatctttgccaacaccggtcacttcaagggaaatgttgtcgccatcaaacatgtgaataagaagcgcattgagctgacccggcaggttctgtttgaactcaaacatatgagagatgttcagttcaaccatctcactcgcttcattggcgcctgcatagaccctcccaacatttgcattgtcactgaatactgtcctcgtgggagtttacaggatattctagaaaatgacagcatcaacttggactggatgtttcgttattcactcattaatgaccttgttaagggcatggcctttctccacaacagcattatttcatcgcatgggagtctcaagtcctccaactgtgtggtggatagtcgttttgtgctcaaaatcacagactatggcctggccagcttccgatcaactgctgaacctgatgacagccatgccctctatgccaagaagctgtggactgccccagaactgctcagtgggaaccccttgccaaccacaggcatgcagaaggctgacgtctatagctttgggatcatcctgcaggagatagcacttcgcagtggtcctttctacttggagggcctggacctcagccccaaagagattgtccagaaggtacgaaatggtcagcggccatatttccggccaagcattgaccggacccaactgaatgaagagctagttttgctgatggagcgatgttgggctcaggacccagctgagcggccagactttggacagattaagggcttcattcggcgctttaacaaggagggtggcaccagcatattggacaacctcctgctgcgcatggaacagtatgccaataacttggagaagctggtggaggaacgcacacaggcctatctggaggaaaaacgcaaggctgaagctctgctctaccaaatcctaccccattcagtggcagagcagttaaaacggggagagactgtacaggctgaggcctttgacagtgttaccatctacttcagtgacattgttggcttcacagcattgtcagcagagagcacccccatgcaggtagtgacacttcttaatgacctgtatacctgctttgatgccataattgacaactttgatgtctacaaggtggagacgattggggatgcttacatggtggtatctggcctcccaggccgaaatggtcaacgccatgcaccagaaattgctcgtatggccctagcattactagatgcagtttcttcctttcgcatccgccaccgaccccatgaccagctgaggctacgcataggggtccatactgggccagtctgtgctggggttgttggcctgaagatgccccgttattgtctttttggagacacagtgaacactgcttctcgaatggagtctaatggtcaagcgctgaagatccatgtctcctctaccaccaaggatgccctagatgagctaggatgcttccagctagagcttcggggggatgtggaaatgaagggaaaaggaaagatgcgaacatactggctcttaggagagcggaaaggacctcctggactcctgtaa

SEQ ID NO: 6 - DNA, 3312 bp, Homo sapiens
atgaccgcctgcgcccgccgagcgggtgggcttccggaccccgggctctgcggtcccgcgtggtgggctccgtccctgccccgcctcccccgggccctgccccggctcccgctcctgctgctcctgcttctgctgcagccccccgccctctccgccgtgttcacggtgggggtcctgggcccctgggcttgcgaccccatcttctctcgggctcgcccggacctggccgcccgcctggccgccgcccgcctgaaccgcgaccccggcctggcaggcggtccccgcttcgaggtagcgctgctgcccgagccttgccggacgccgggctcgctgggggccgtgtcctccgcgctggcccgcgtgtcgggcctcgtgggtccggtgaaccctgcggcctgccggccagccgagctgctcgccgaagaagccgggatcgcgctggtgccctggggctgcccctggacgcaggcggagggcaccacggcccctgccgtgacccccgccgcggatgccctctacgccctgcttcgcgcattcggctgggcgcgcgtggccctggtcaccgccccccaggacctgtgggtggaggcgggacgctcactgtccacggcactcagggcccggggcctgcctgtcgcctccgtgacttccatggagcccttggacctgtctggagcccgggaggccctgaggaaggttcgggacgggcccagggtcacagcagtgatcatggtgatgcactcggtgctgctgggtggcgaggagcagcgctacctcctggaggccgcagaggagctgggcctgaccgatggctccctggtcttcctgcccttcgacacgatccactacgccttgtccccaggcccggaggccttggccgcactcgccaacagctcccagcttcgcagggcccacgatgccgtgctcaccctcacgcgccactgtccctctgaaggcagcgtgctggacagcctgcgcagggctcaagagcgccgcgagctgccctctgacctcaatctgcagcaggtctccccactctttggcaccatctatgacgcggtcttcttgctggcaaggggcgtggcagaagcgcgggctgccgcaggtggcagatgggtgtccggagcagctgtggcccgccacatccgggatgcgcaggtccctggcttctgcggggacctaggaggagacgaggagcccccattcgtgctgctagacacggacgcggcgggagaccggctttttgccacatacatgctggatcctgcccggggctccttcctctccgccggtacccggatgcacttcccgcgtgggggatcagcacccggacctgacccctcgtgctggttcgatccaaacaacatctgcggtggaggactggagccgggcctcgtctttcttggcttcctcctggtggttgggatggggctggctggggccttcctggcccattatgtgaggcaccggctacttcacatgcaaatggtctccggccccaacaagatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgttcaagaacatcaacaagggccggaagacgaacatcattgactcgatgcttcggatgctggagcagtactctagtaacctggaggatctgatccgggagcgcacggaggagctggagctggaaaagcagaagacagaccggctgcttacacagatgctgcctccgtctgtggctgaggccttgaagacggggacaccagtggagcccgagtactttgagcaagtgacactgtactttagtgacattgtgggcttcaccaccatctctgccatgagtgagcccattgaggttgtggacctgctcaacgatctctacacactctttgatgccatcattggttcccacgatgtctacaaggtggagacaataggggacgcctatatggtggcctcggggctgccccagcggaatgggcagcgacacgcggcagagatcgccaacatgtcactggacatcctcagtgccgtgggcactttccgcatgcgccatatgcctgaggttcccgtgcgcatccgcataggcctgcactcgggtccatgcgtggcaggcgtggtgggcctcaccatgccgcggtactgcctgtttggggacacggtcaacaccgcctcgcgcatggagtccaccgggctgccttaccgcatccacgtgaacttgagcactgtggggattctccgtgctctggactcgggctaccaggtggagctgcgaggccgcacggagctgaagggcaagggcgccgaggacactttctggctagtgggcagacgcggcttcaacaagcccatccccaaaccgcctgacctgcaaccggggtccagcaaccacggcatcagcctgcaggagatcccacccgagcggcgacggaagctggagaaggcgcggccgggccagttctcttga

SEQ ID NO: 7 - DNA, 2799 bp, Homo sapiens
atggagggggtgggcttgtggcccccacagtacgggtccccagaggctcagcggaagccagggctgcaggccctgaagtccatttgtggtgctgtggatgcactcggcggtgctggggggcttggagatgaccaccctgctgagccgtgtgcctgggccgagggcctggtggatgggagattagtcttcctgccctatgacatgctgctcttctccctgtcctactgcaaccgctcctacctgtctctcggcactggtggacccctgcaggaggcctatgatgcagtgctcaccatcagccaggagtcgggccccatagacaaggcctttgctgctgcccggcctgatggagaggcggctgcccaccttgagccagagcaggtaggtgtgctccttgtgcagaccccgaatgccttgagtaggggtccggggcaggggtgcacgcggtcaggactcagtgaacttgggtctcctggggatgcccgtttcttggtgttaagaagacagcagtgcctggaggtggtgacggatgtggatgctgatggactcttggcaacttgggaacgttgcgactggggaccagcagatgcatcaggcccaggggttccagacccgagcccgaggcagagggcctgtgtgaagacagtgttcagtgccctcctgacccagctgggactgacagtgagacagaaatggaaacggggagaggaaagtttgggcatccaccagctgcagctggtgtggggcccccaccggatcctgctgacagcccaggagctcaccttcatccatccacccctgaacagacagaggctgcatgtggatggtgaatggagaagtgtggcggatggtgggagcctgagatgctttgtgctcaaggtcactgaccatgagctgctatggacagctcctgagctgctgccggggcctgggcgccctgggcggcgcaccctcacaggggacatcttcagcactggcatcatcctgcaggaggtgctgactcgggcccactctactgctcctcgggacttccagtggaagaatcataatgatgatgataatgataagcaggcagtgtcgttcacacctgtggggaaccggcacgccagtgtctgcgtggcaaggctggaggaaagagctgcgctgtcaaggtttcaagatcattggtgggcattgttggcaacagagcagcggcagttgttgcggggcctgagggtgggggagagtctgccaggaccccaggcccgccccatggtgttggttgccgaaatcatcaggagggtggcatctccccctcctctgtgccggccgctgaagtgctgggacgaggctccagaggacagacctagcatggaccagatctacagccagttcaaaagcatcaaccaaggcaagaggaccagtgttgctgactccatgctgtggttgctggagaaatattcccagaacctggaggacctgattcaggagcagactgaggaactggagctgaagagagagaagacagaaaggctgctctgtcagatgattcccccgtctgtggctgaagctcggaaaatgggggcaactgtggaaccagggtattttgaccaggttaccatatacttcagtgacattgtgggtttcaccatcatctcagccctgagtgaacccattgaggcggtgggcttgctcaacgatctctacatgctgtttgatgctgttctgggcagccatgacctgtataaggtggagaccattggggacgcctacatggtggtgtgggggctgcctcagtgcaatggcagtcagcatgcggccgagatcaataacatggctctggatatccttggctctgtgggtgacttccggaagaggcatgcacccaacgtgcccatttgcatcagggctggcctgcattcagacctgctggagctcactgggtacattcgagtttcagggccctgtgcagcaggggtcgtgggtctcacgatgccttggtattgcctctctggggacactgtcaacacggcttcccagatggagtccacagggctgcatgagtatctcacacttctatcctgcccggggagagactctttcaataaccttcctgacaccaccatgaagacatccccagcgtttttggcccttactggcagcaggagctaccatgatggcttggctagagcagctaagatgttgaagccccaggtcctctgcctctggctgtcatacagaattcatgtcagccaaagcactgtccagatactgctcagccttgatgaaagctaccaaattgatatcagaggggaagggcatcgaagagacctactgggtggggggaaggcaggcttccccaggcccctccccgcacctgtggacatcagacctggctctggctgctcactcaatggttccttctggggcttcacagcaccacggattgcaaagctccagcaacccttgaaacctggtcctccagcatccccagtcagacagcccaacaccatgccaagctcctggcgtctccgcacctacccctatccattagcaagtctggagcctccagagcctctggggaggcagaacaggggtgaggacagcaccttccagctctccaccacctccacaatcagtcatcaagtcctgtgcggcctgccacgatggtctccgacttcggtccacaacaagggcaccaggcttcatgctgctctcttgcctctattcccacccctaatctacctgcaacgcagaccttgcgacgtcacttttcgacttagcaagtgtgcactactgatgaccaagggcctttgctga

SEQ ID NO: 8 - DNA, 3327 bp, Homo sapiens
atgttcctgggactcgggcgcttttctcgccttgttctctggtttgcggctttcaggaaactgctgggacaccatggccttgcatctgccaagttcctgtggtgcttgtgccttctgtctgtcatgtcccttccgcagcaggtgtggacactcccctacaagataggggtggtgggcccttgggcttgtgattcgctgttttcaaaggccctgcctgaggttgctgcgcgattagccattgagcgaatcaaccgggacccatcttttgacctgagttattcttttgaatacgtgattctcaatgaagactgccagacttcgagggctctctccagtttcatttcccaccaccagatggcctcaggatttattggacctaccaaccctggctactgcgaggcagcctcgctcctgggaaacagctgggacaaaggaattttctcttgggcttgtgtgaattatgaattagacaataaaattagctacccgaccttttctcggacactcccttctcccatccgggtgcttgtaactgtcatgaaatatttccagtgggctcatgctggagtcatttcctcagatgaagacatttgggtgcatacagccaatcgagtcgcaagtgctcttcggagccacggcttacctgtaggggtcgtcctgaccacaggacaagacagccaaagcatgcggaaagccctccagaggattcaccaggcagacagaattcgcataatcatcatgtgtatgcattcagctttgattgggggagagactcagatgcatctcttggaatgtgctcatgatctgaaaatgactgatggaacctacgtctttgttccttatgatgccctgctctacagtttaccttataagcacaccccctaccgggtcctaaggaacaacccaaagctccgggaagcctatgatgcagtgttgaccattacagtggagtcccaagaaaagaccttctatcaagccttcacagaggcagcagcaagaggtgaaattcctgagaagctggagttcgatcaagtttcaccgttgtttggaaccatctacaattcaatttactttatcgcacaagccatgaataatgctatgaaagaaaatggacaggctggtgctgccagcctggttcagcattccagaaacatgcagttccatggattcaaccagttgatgaggacagattcaaatggaaatggaatttcagaatatgtaatcctggacaccaacttgaaagaatgggaactccatagcacctacactgtggacatggaaatggagctgctacgtttcggagggacccctattcacttccctggtggcaggccccctagagcagatgcaaaatgctggtttgcagaagggaagatctgccatggaggcatcgaccctgcctttgccatgatggtctgccttactttgcttatagccctgctgtctattaatggatttgcttactttataaggcgtcgtataaataaaatccagttgatcaaaggacccaatagaattctactgactttggaggatgtaacgtttatcaatccccactttggcagtaagagaggaagtcgtgccagtgtaagcttccagattacctcagaggtccaaagtgggaggtccccaagactctccttttcttcagggagtctaactccagctacctatgaaaactccaacatagcgatttatgagggtgattgggtgtggctgaaaaagttctcccttggagattttggagaccttaagtccatcaaatcaagagcaagtgatgtgttcgaaatgatgaaggacttgcgtcatgagaatattaaccctttattgggtttcttctatgattcggggatgtttgccattgtgacagaattctgttcccgagggagcctagaagacatactgacaaatcaagatgtgaaacttgactggatgtttaaatcatcactcttgctggatctcataaagggcatgaagtacttacaccacagagagtttgttcatgggaggctaaagtctcgaaactgtgtggtagatgggcgttttgtactaaaagtgacagattatggctttaacgacatcttagaaatgctgagactctctgaagaggaatcttctatggaagagctgctgtggacggcccctgaactgttgagagctccaagaggcagcaggttaggttcttttgcaggagatgtctatagctttgccatcatcatgcaagaagtgatggtccggggtaccccattctgcatgatggatctgccagctcaagaaatcataaacagacttaagaagcctcctcctgtgtacagaccagtagttcctcctgagcatgcccctccagaatgtctccagctgatgaagcagtgctgggctgaggctgcagaacaacgaccaacttttgatgaaatatttaaccagtttaaaacttttaataaagggaagaagaccaatattattgattctatgcttcggatgttggagcaatattctagcaacttggaagatttgattcgggagcggactgaagagctggaaattgaaaaacagaaaacggaaaagcttctaacacagatgctaccaccatcagttgctgaatctctcaaaaagggctgcacagttgaacctgagggctttgacttggtcaccttgtacttcagcgacattgtgggcttcacaaccatttcagccatgagtgagcccattgaggtcgtggatcttctgaatgacctgtacacactctttgatgcaataattggcagtcatgatgtctacaaggtagagaccattggagatgcctacatggtggcttcaggcctcccaaagaggaatggcagtaggcatgcagctgagattgcaaacatgtccttagatatcctgagctctgtgggcactttcaagatgcggcacatgccagaagtgccggtccgaattcgaattggccttcactcagggccggttgttgctggagtggtgggcctcaccatgcccagatactgcttgtttggagacactgtgaacacagcttctcggatggaatctacaggcttaccttatcgcattcatgtcagtctcagcactgttacaattcttcaaaatctgagtgagggctatgaagtggagcttcgaggaagaacagagctcaagggcaaaggcacagaggaaaccttctggctgattgggaaaaaaggcttcatgaagccccttcctgtgcccccaccagtggacaaagatgggcaagtgggccatggcctgcaaccagtggagattgcagccttccaaagaagaaaagcagaaaggcagttggtgagaaacaagccataa

SEQ ID NO: 9 - DNA, 1269 bp, Homo sapiens
atgccctggtcgggtaccccgcaaggagatgtttacagcttcgccattctgatgagggagctgatctaccattgggaccacgggccttttgatgacctccacgaggcaccggatgaaatcatcaaccaaatcaaagaccctgcagcagcagtcccactgcaaccttccctgcccgaggagaagggcaatgaaaagatcgtggccatggtgagggtgtgttgggatgaatctctggagaaaagacccagtttctcttccatcaagaaaactttacgagaggccagtcccagaggtcatgtgagcatactggccagtgtgatgagcaagctggaagtgtatgccaattacctggaggaagtggtgcaagagaggaccagccagctgactgcagagaagaggaaggtggagaagcttctgtccaccaaagtgcccagcttcactggagaacaactactagccggaaggtccgtggaaccagaacatttcgaatctgtgacaatctttttatctgatattgttggattcacaaagctgtgttctctcagctcccctttgcaagtcgtcaagctcctcaatgacgtgtacagtttattcgatcacatcatcacaacttatgatgtttataaggttgagaccattggagatgcatacatggtggctagtggactccctaccacaatggaagccagcatgtggctgagattgccaccatgtccctgcacttcctcagtgccaccatctgcttccaaattgggcacatgccccaggagaagctccagcttcgtattggcctctacacaggtcctgtggtggctggtgtgctggggattaccatgtccagatactgtctatttggagacactgtcaacatggcatccagaatggagagcagcagttcacctctccggattcatgtctctcagagcactgcaagcaccctggtggcattgggagggtgtttgctcccagcctgtgcctccagcaaatgcagatttggaggagctgaagatggtgaactcatttcctggggtgccagggctgacttgtggctctcagccctgtgcaactgccctagctgccagatatttacagcccattaacttgagaatttgtgttgaacccagtttcattctgcaggaggggaaagtggagaggatgaagcctgtctttcttgtgcaagatgcctgctctctaacctttggtgctgattttccttatcagctaatcagcttgcaaggagataggagctcatatctgacagcatcttga

SEQ ID NO: 10 - DNA, 1623 bp, Homo sapiens
atgccgtctctgctggtgctcactttctccccgtgcgtactactcggctgggcgttgctggccggcggcaccggtggcggtggcgttggcggcggcggcggtggcgcgggcataggcggcggacgccaggagagagaggcgctgccgccacagaagatcgaggtgctggtgttactgccccaggatgactcgtacttgttttcactcacccgggtgcggccggccatcgagtatgctctgcgcagcgtggagggcaacgggactgggaggcggcttctgccgccgggcactcgcttccaggtggcttacgaggattcagactgtgggaaccgtgcgctcttcagcttggtggaccgcgtggcggcggcgcggggcgccaagccagaccttatcctggggccagtgtgcgagtatgcagcagcgccagtggcccggcttgcatcgcactgggacctgcccatgctgtcggctggggcgctggccgctggcttccagcacaaggactctgagtactcgcacctcacgcgcgtggcgcccgcctacgccaagatgggcgagatgatgctcgccctgttccgccaccaccactggagccgcgctgcactggtctacagcgacgacaagctggagcggaactgctacttcaccctcgagggggtccacgaggtcttccaggaggagggtttgcacacgtccatctacagtttcgacgagaccaaagacttggatctggaagacatcgtgcgcaatatccaggccagtgagagagtggtgatcatgtgtgcgagcagtgacaccatccggagcatcatgctggtggcgcacaggcatggcatgaccagtggagactacgccttcttcaacattgagctcttcaacagctcttcctatggagatggctcatggaagagaggagacaaacacgactttgaagctaagcaagcatactcgtccctccagacagtcactctactgaggacagtgaaacctgagtttgagaagttttccatggaggtgaaaagttcagttgagaaacaagggctcaatatggaggattacgttaacatgtttgttgaaggattccacgatgccatcctcctctacgtcttggctctacatgaagtactcagagctggttacagcaaaaaggatggagggaaaattatacagcagacttggaacagaacatttgaaggtatcgccgggcaggtgtccatagatgccaacggagaccgatatggggatttctctgtgattgccatgactgatgtggaggcgggcacccaggaggttattggtgattattttggaaaagaaggtcgttttgaaatgcggccgaatgtcaaatatccttggggccctttaaaactgagaatagatgaaaaccgaattgtagagcatacaaacagctctccctgcaaatcatgtggcctagaagaatcggcagtgacaggaattgtcgtgggggctttactaggagctggcttgctaatggccttctactttttcaggaagaaatacagaataaccattgagaggcgaacccagcaagaagaaagtaaccttggaaaacatcgggaattacgggaagattccatcagatcccatttttcagtagcttaa

SEQ ID NO: 11 - DNA, 34 bp, Artificial Sequence
  Description of Artificial Sequence Synthetic untranslated oligonucleotide
gccagtcccagttcctgtgccttaagaacctcgc

(19) United States
(12) Patent Application Publication
(10) Pub. No.: US 2012/0028278 A1
(43) Pub. Date: Feb. 2, 2012

(54) CELL LINES EXPRESSING GUANYLATE CYCLASE-C AND METHODS OF USING THEM

(75) Inventors: Kambiz Shekdar, New York, NY (US); Jessica Langer, Highland Park, NJ (US)

(73) Assignee: CHROMOCELL CORPORATION, North Brunswick, NJ (US)

(21) Appl. No.: 13/147,325

(22) PCT Filed: Feb. 1, 2010
(86) PCT No.: PCT/US2010/000280

§ 371 Date: Aug. 1, 2011

Related U.S. Application Data
(60) Provisional application No. 61/149,309, filed on Feb. 2, 2009.

(51) Int. Cl. C12N 5/10; G01N 33/53; C12Q 1/02; C12N 15/85; C12Q 1/25

(52) U.S. Cl. 435/7.92; 435/325; 435/366; 435/455; 435/29

(57) ABSTRACT

Cell lines that stably express GC-C and methods for using those cell lines are disclosed herein. The invention includes cell lines that express GC-C and techniques for creating cell lines. The GC-C-expressing cell lines are highly sensitive, physiologically relevant and produce consistent results in cell-based assays, e.g., high throughput screening assays.

This application claims the benefit of U.S. Provisional Application No. 002298-022-001 filed Feb. 2, 2009 which is incorporated by reference herein in its entirety.

### FIELD OF THE INVENTION

The invention relates to guanylate cyclase-C (“GC-C”) and cells and cell lines stably expressing GC-C. The invention further provides methods of making such cells and cell lines. The GC-C-expressing cells and cell lines provided herein are useful in identifying GC-C modulators.

### BACKGROUND

The single membrane spanning guanylyl cyclase family includes guanylate cyclases A-G. Members of this family are characterized by an extracellular ligand binding domain, a transmembrane domain and an intracellular kinase homology domain followed by a catalytic domain. There exist guanylyl cyclases that contain no transmembrane domains and are soluble (such as the nitric oxide receptor) as well as those that contain multiple transmembrane domains. Additionally, NPR3 is a related receptor that lacks the guanylyl cyclase domain.

Guanylate cyclase-A (GC-A, or NPR1) binds the endogenous ligands atrial natriuretic peptide and B-type natriuretic peptide and is involved in maintaining blood pressure by controlling vasodilation, salt and water excretion. Guanylate cyclase-B (GC-B, or NPR2) binds C-type natriuretic peptide and is involved in tissue remodeling that follows vascular injury and inflammation. Guanylate cyclase-D and -G (GC-D and GC-G) have been characterized as pseudogenes in humans. However, GC-D is expressed in mice and plays an olfactory role. Guanylate cyclase-E and -F (GC-E and GC-F) regulate the dark cycle of phototransduction in the retina. GC-E and -F are activated by guanylyl cyclase-activating proteins (GCAPs), as opposed to extracellular ligands.

Guanylate cyclase-C (GC-C) is expressed primarily on the apical surface of intestinal epithelial cells that regulate chloride secretion by the cystic fibrosis transmembrane conductance regulator (CFTR). GC-C is also expressed in the kidney, testis, liver, placenta and lung. GC-C is thought to form trimers or higher order multimers. GC-C is both tyrosine- and serine-phosphorylated as well as glycosylated. Glycosylation is required for ligand-mediated activation. Ligands of GC-C include the endogenous peptides guanylin, lymphoguanylin and uroguanylin and the bacterially derived heat-stable enterotoxin STa. Binding of any of the four peptides to GC-C results in increased levels of cyclic guanosine monophosphate (cGMP) in the cell and stimulates water and chloride secretion. GC-C and its ligands are important clinical targets for managing a variety of gastrointestinal conditions, including irritable bowel syndrome, forms of diarrhea and forms of constipation, as well as colon cancer. For example, GC-C over-expression is a sensitive biomarker for colon adenocarcinomas. GC-C and its ligands may also be useful in managing kidney conditions.

The discovery of new and improved therapeutics that specifically target GC-C and other guanylyl cyclase family members has been hampered by the lack of robust, physiologically relevant cell-based systems and more especially cell-based systems that are amenable to high through-put formats for identifying and testing modulators of GC-C and other guanylyl cyclase family members. Cell-based systems are preferred for drug discovery and validation because they provide a functional assay for a compound as opposed to cell-free systems, which only provide a binding assay. Moreover, cell-based systems have the advantage of simultaneously testing cytotoxicity. Ideally, cell-based systems should also stably and constitutively express the target protein. It is also desirable for a cell-based system to be reproducible. Cell lines that naturally express endogenous GC-C possess drawbacks because it is unclear what proportion of the signal in cell-based assays using these cell lines are attributable to GC-C versus other endogenously expressed targets that contribute to the assay response. A need exists for cell lines that express GC-C in isolation from other factors. The present invention addresses these problems.

### SUMMARY OF THE INVENTION

We have discovered new and useful cells and cell lines that express functional guanylate cyclase C (GC-C). These cells and cell lines are useful in cell-based assays, in particular high throughput assays to study the functions of GC-C and to screen for GC-C modulators.

In one aspect, the invention provides a cell or cell line engineered to stably express GC-C (for example, a mammalian or human GC-C). The GC-C may be a functional GC-C or various GC-C variants. In one embodiment, the GC-C is expressed from an introduced nucleic acid encoding it. In another embodiment, the GC-C is expressed from an endogenous nucleic acid engineered by gene activation. In a further embodiment, the GC-C does not comprise any polypeptide tag. The cells may be primary or immortalized cells, and may be cells of, for example, primate (e.g., human or monkey), rodent (e.g., mouse, rat, or hamster), or insect (e.g., fruit fly) origin. The cells may optionally not express endogenous GC-C prior to engineering. In one embodiment, the cells or cell lines of the invention are derived from 293T cell(s).

In another aspect, the invention provides a GC-C expressing cell or cell line that produces a Z′ factor of at least 0.4, at least 0.45, at least 0.5, at least 0.55, at least 0.6, at least 0.65, at least 0.7, at least 0.75, at least 0.8, or at least 0.85 in an assay, such as a competitive ELISA that measures cGMP levels. The cell or cell line may be maintained without selective pressure or grown in the absence of selective pressure. In one embodiment, the cell or cell line expresses the GC-C in the absence of selective pressure for at least 15 days, at least 30 days, at least 45 days, at least 60 days, at least 75 days, at least 100 days, at least 120 days, or at least 150 days. In another embodiment, the cell or cell line expresses the GC-C at a consistent level in the absence of selective pressure for at least 15 days, at least 30 days, at least 45 days, at least 60 days, at least 75 days, at least 100 days, at least 120 days, or at least 150 days.

In a further aspect, the cells and cell lines of the invention are suitable for use in a high throughput screening assay. In one embodiment, the cells and cell lines of the invention produce a detectable signal-to-noise ratio. The signal-to-noise ratio may be greater than 1 (for example, in response to an agonist or antagonist of GC-C).

The invention also provides a cell or cell line wherein the GC-C is selected from the group consisting of: a) a GC-C polypeptide comprising the amino acid sequence set forth in SEQ ID NO: 3; b) a GC-C polypeptide comprising an amino acid sequence that is at least 95% identical to the amino acid sequence of SEQ ID NO: 3; c) a GC-C polypeptide encoded by a nucleic acid that hybridizes under stringent condition to SEQ ID NO: 2; and d) a GC-C polypeptide that is an allelic variant of any one of SEQ ID NO: 3. The invention further provides a cell or cell line wherein the GC-C is encoded by a nucleic acid selected from the group consisting of: a) a nucleic acid comprising the sequence set forth in SEQ ID NO: 3; b) a nucleic acid that hybridizes to a nucleic acid comprising the nucleotide sequence of any one of SEQ ID NO: 2 under stringent conditions; c) a nucleic acid that encodes a polypeptide comprising the amino acid sequence of any one of SEQ ID NO: 3; d) a nucleic acid comprising a nucleotide sequence that is at least 95% identical to any one of SEQ ID NO: 2; and e) a nucleic acid that is an allelic variant of any one of SEQ ID NO: 2.

In another aspect, the invention provides a collection of cells or cell lines, wherein the cells or cell lines in the collection express different forms of GC-C. In one embodiment, the different forms of GC-C comprise at least one single nucleotide polymorphism (SNP). In another embodiment, at least one cell or cell line in the collection expresses an introduced receptor other than GC-C. For example, at least one cell or cell line may express an introduced NPR3 or a guanylyl cyclase other than GC-C. In a further embodiment, the cells or cell lines in the collection are matched to share the same physiological property (for example, cell type, metabolism, cell passage (age), growth rate, adherence to a tissue culture surface, Z′ factor or expression level of GC-C) to allow parallel processing and accurate assay readouts. These can be achieved by generating and growing the cells and cell lines under identical conditions, achievable by, e.g., automation.

In one embodiment, the invention provides a method for producing GC-C-expressing cells or cell lines or collections of cells or cell lines, comprising the steps of: (a) introducing into host cells a nucleic acid encoding GC-C; (b) introducing into the host cells a molecular beacon that detects the expression of GC-C into the host cells; and (c) isolating a cell that expresses GC-C. In another embodiment, the method comprises the steps of: (a) introducing into host cells one or more nucleic acid sequences that activate expression of endogenous GC-C; (b) introducing into the host cells a molecular beacon that detects expression of the activated GC-C; and (c) isolating cells that express the activated GC-C. The methods for producing GC-C-expressing cells or cell lines may further comprise the step of generating a cell line from the cell isolated in step (c). The host cells used for the methods for producing GC-C-expressing cells or cell lines may be mammalian cells. The GC-C used for the methods for producing GC-C-expressing cells or cell lines may comprise the amino acid sequence set forth in SEQ ID NO: 3 or may be encoded by a nucleic acid comprising SEQ ID NO: 2. In one embodiment, the isolating step of these methods utilizes a fluorescence activated cell sorter. Utilizing the methods of the invention, the GC-C expressing cells or cell lines of the collection may be produced in parallel.

In another aspect, the invention provides a method for identifying a modulator of a GC-C (for example, a human GC-C) function, comprising the step of exposing the cell or cell line of the invention to a test compound and detecting a change in a GC-C function in a cell compared to a cell not contacted with the test compound, wherein a change in said function indicates that the test compound is a GC-C modulator. The modulator may be a GC-C antagonist or a GC-C agonist. In one embodiment, the detecting step utilizes an assay for cGMP level or guanylyl cyclase activity. The test compound may be a small molecule, a chemical moiety, a polypeptide or an antibody. In one embodiment, the test compound is a library of compounds (such as a small molecule library, a combinatorial library, a peptide library or an antibody library).

In a further aspect, the invention provides a method for identifying a modulator of any introduced protein, comprising the step of exposing a collection of cells or cell lines of the invention to a test compound and detecting a change in the function of the introduced protein in a cell compared to a cell not contacted with the test compound, wherein a change in said function indicates that the test compound is a modulator of the introduced protein. In one embodiment, the modulator affects the function of all the introduced proteins in the collection. For example, the modulator is either an agonist of all the introduced proteins in the collection or an antagonist of all the introduced proteins in the collection. In another embodiment, the modulator affects the function of a subset of the introduced proteins in the collection. For example, the modulator is an agonist of the subset of the introduced proteins in the collection or an antagonist of the subset of the introduced proteins in the collection. In a further embodiment, the modulator is an agonist of some of the subset of introduced proteins in the collection and an antagonist of the remaining subset of introduced proteins in the collection.

In a further aspect, the invention provides a modulator of GC-C identified by any of the methods of the invention.

In another aspect, this invention provides a cell engineered to stably express a GC-C at a consistent level over time, the cell made by a method comprising the steps of: a) providing a plurality of cells that express mRNA encoding the GC-C; b) dispersing the cells individually into individual culture vessels, thereby providing a plurality of separate cell cultures; c) culturing the cells under a set of desired culture conditions using automated cell culture methods characterized in that the conditions are substantially identical for each of the separate cell cultures, during which culturing the number of cells per separate cell culture is normalized, and wherein the separate cultures are passaged on the same schedule; d) assaying the separate cell cultures to measure expression of the GC-C at least twice; and e) identifying a separate cell culture that expresses the GC-C at a consistent level in both assays, thereby obtaining said cell.

### BRIEF DESCRIPTION OF THE FIGURES

FIG. 1 is a bar graph depicting the results of a competitive ELISA for detection of cGMP. The “Clone” value was generated by assaying cell lysates from a produced GC-C-expressing cell line of this invention, treated with 100 nM guanylin. The “Control” value was generated by performing the ELISA using no cell lysate.

FIG. 2 depicts a guanylin competitive dose-response curve. The produced GC-C-expressing cell line was exposed to increasing concentrations of guanylin for 40 min at 37° C. Cellular cGMP was measured using a competitive ELISA, with the assay response shown on the y-axis. Each data point is a mean of triplicates. Concentrations of guanylin are shown on the x-axis.

### DETAILED DISCLOSURE

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. The materials, methods, and examples are illustrative only and not intended to be limiting.

In order that the present invention may be more readily understood, certain terms are first defined. Additional definitions are set forth throughout the detailed description.

The term “stable” or “stably expressing” is meant to distinguish the cells and cell lines of the invention from cells with transient expression as the terms “stable expression” and “transient expression” would be understood by a person of skill in the art.

The term “cell line” or “clonal cell line” refers to a population of cells that are all progeny of a single original cell. As used herein, cell lines are maintained in vitro in cell culture and may be frozen in aliquots to establish banks of clonal cells.

The term “stringent conditions” or “stringent hybridization conditions” describe temperature and salt conditions for hybridizing one or more nucleic acid probes to a nucleic acid sample and washing off probes that have not bound specifically to target nucleic acids in the sample. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. An example of stringent hybridization conditions is hybridization in 6×SSC at about 45° C., followed by at least one wash in 0.2×SSC, 0.1% SDS at 60° C. A further example of stringent hybridization conditions is hybridization in 6×SSC at about 45° C., followed by at least one wash in 0.2×SSC, 0.1% SDS at 65° C. Stringent conditions include hybridization in 0.5M sodium phosphate, 7% SDS at 65° C., followed by at least one wash at 0.2×SSC, 1% SDS at 65° C.

The phrase “percent identical” or “percent identity” in connection with amino acid and/or nucleic acid sequences refers to the similarity between at least two different sequences. This percent identity can be determined by standard alignment algorithms, for example, the Basic Local Alignment Tool (BLAST) described by Altshul et al. ((1990) J. Mol. Biol., 215: 403-410); the algorithm of Needleman et al. ((1970) J. Mol. Biol., 48: 444-453); or the algorithm of Meyers et al. ((1988) Comput. Appl. Biosci., 4: 11-17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) that has been incorporated into the ALIGN program (version 2.0), using a PAM120 weight residue table, a gap length penalty of 12 and a gap penalty of 4. The percent identity is usually calculated by comparing sequences of similar length. Protein analysis software matches similar sequences using measures of similarity assigned to various substitutions, deletions and other modifications, including conservative amino acid substitutions. For instance, the GCG Wisconsin Package (Accelrys, Inc.) contains programs such as “Gap” and “Bestfit” that can be used with default parameters to determine sequence identity between closely related polypeptides, such as homologous polypeptides from different species of organisms or between a wild type protein and a mutant thereof. See, e.g., GCG Version 6.1. Polypeptide sequences also can be compared using FASTA using default or recommended parameters. A program in GCG Version 6.1. FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). The length of polypeptide sequences compared for identity will generally be at least about 16 amino acid residues, usually at least about 20 residues, more usually at least about 24 residues, typically at least about 28 residues, and preferably more than about 35 residues. The length of a DNA sequence compared for identity will generally be at least about 48 nucleic acid residues, usually at least about 60 nucleic acid residues, more usually at least about 72 nucleic acid residues, typically at least about 84 nucleic acid residues, and preferably more than about 105 nucleic acid residues.

The phrase “substantially as set out,” “substantially identical” or “substantially homologous” in connection with an amino acid nucleotide sequence means that the relevant amino acid or nucleotide sequence will be identical to or have insubstantial differences (through conserved amino acid substitutions) in comparison to the sequences that are set out. Insubstantial differences include minor amino acid changes, such as 1 or 2 substitutions in a 50 amino acid sequence of a specified region.

The terms “potentiator”, “agonist” or “activator” refer to a compound or substance that activates a biological function of GC-C.

The terms “inhibitor”, “antagonist” or “blocker” refers to a compound or substance that decreases a biological function of GC-C.

The term “modulator” refers to a compound or substance that alters a structure, conformation, biochemical or biophysical property or functionality of a GC-C either positively or negatively. The modulator can be a GC-C agonist (potentiator or activator) or antagonist (inhibitor or blocker), including partial agonists or antagonists, selective agonists or antagonists and inverse agonists, and can be an allosteric modulator. A substance or compound is a modulator even if its modulating activity changes under different conditions or concentrations or with respect to different forms of GC-C. As used herein, a modulator may affect the guanylyl cyclase activity of GC-C, the response of GC-C to another regulatory compound or the selectivity of GC-C. A modulator may also change the ability of another modulator to affect a function of GC-C.

The phrase “functional GC-C” refers to a GC-C that behaves in substantially the same way as GC-C in a cell that naturally expresses endogenous GC-C without engineering, e.g., by responding to a known activator (e.g., guanylin, lymphoguanylin, uroguanylin, STa, linaclotide (Microbia, Inc.) or SP-304 (Guanilib; Callisto Pharmaceuticals)), or a known inhibitor (e.g., 5-(3-bromophenyl)-1,3-dimethyl-5,11-dihydro-1H-indeno[2′,1′:5,6]pyrido[2,3-d]pyrimidine-2,4,6-trione (BPIPP), or Tyrphostin A25 (AG82)). GC-C behavior can be determined by, for example, physiological activities and pharmacological responses. Physiological activities include, but are not limited to, guanylyl cyclase activity, effect on cGMP levels, stimulation or reduction of water or chloride secretion and regulation of mucosal and/or epithelial fluid absorption or secretion. Pharmacological responses include, but are not limited to, activation by guanylin, lymphoguanylin, uroguanylin, STa, linaclotide or SP-304; or inhibition by 5-(3-bromophenyl)-1,3-dimethyl-5,11-dihydro-1H-indeno[2′,1′:5,6]pyrido[2,3-d]pyrimidine-2,4,6-trione (BPIPP), or Tyrphostin A25 (AG82).

A “heterologous” or “introduced” GC-C protein means that the GC-C protein is encoded by a polynucleotide introduced into a host cell.

This application relates to novel cells and cell lines that have been engineered to express GC-C (e.g., SEQ ID NO: 3). According to the invention, the GC-C can be from any mammal, including rat, mouse, rabbit, goat, dog, cow, pig or primate (e.g., human). The expressed GC-C may affect the levels of cGMP and may be modulated by, for example, guanylin, lymphoguanylin, uroguanylin, STa, linaclotide, SP-304, 5-(3-bromophenyl)-1,3-dimethyl-5,11-dihydro-1H-indeno[2′,1′:5,6]pyrido[2,3-d]pyrimidine-2,4,6-trione (BPIPP), or Tyrphostin A25 (AG82). In some embodiments, the novel cells or cell lines of the invention express an introduced functional GC-C (e.g., GC-C encoded by a transgene; e.g., SEQ ID NO: 2). In some embodiments, the novel cells or cell lines of the invention express a naturally-occurring GC-C, encoded by an endogenous GC-C gene that has been activated by gene activation technology. In preferred embodiments, the cells and cell lines stably expressed GC-C. The GC-C cells and cell lines of the invention have enhanced properties compared to cells and cell lines made by conventional methods. For example, the GC-C cells and cell lines have enhanced stability of expression (even when maintained in culture without selective pressure such as antibiotics) and possess high Z′ values in cell-based assays.

In other aspects, the invention provides methods of making and using the novel cells and cell lines. In other aspects, the invention provides methods of using the cells and cell lines of this invention to screen for modulators of GC-C, which, for example, may increase or reduce guanylyl cyclase activity or cGMP levels, stimulate or reduce water or chloride secretion or regulate mucosal and/or epithelial fluid absorption or secretion mediated by GC-C. Such modulators are useful in treating diseases and conditions associated with GC-C dysregulation or dysfunction. Non-limiting examples of such diseases and conditions include gastrointestinal conditions and indications associated with irritable bowel syndrome (e.g., IBS-C, IBS-D and IBS-M), bowel cleansing, chronic idiopathic constipation, opioid/drug-induced constipation, bedridden patient and geriatric constipation, infections or acute infectious diarrhea, pediatric diarrhea (e.g., viral, bacterial and protozoan), Travelers' diarrhea (TD), E. coli infection, cholera infection, viral gastroenteritis, rotavirus infection, HIV infection, malabsorption syndromes, short bowel syndrome, colitis (collagenous and lymphocytic), ulcerative colitis (UC), Crohn's Disease, diverticulitis, cystic fibrosis, peptic ulcers; cancers (e.g., colon cancer); kidney conditions; pulmonary indications; cystic fibrosis; cardiac fibrosis; cardiac hypertrophy; hypertension; eye disorders (e.g., autosomal dominant retinitis pigmentosa (ADRP) and Leber congenital amaurosis (LCA)); growth disorders (e.g., short stature); stroke and other vascular injury; central nervous system indications; memory conditions; depression; and inflammatory disorders (e.g., rheumatoid arthritis).

In various embodiments, the cell or cell line of the invention expresses GC-C (e.g., functional GC-C) at a consistent level of expression for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200 days or over 200 days, where consistent expression refers to a level of expression that does not vary by more than: 1%, 2%, 3%, 4%, 5%, 6%, 7%, 8% 9% or 10% over 2 to 4 days of continuous cell culture; 2%, 4%, 6%, 8%, 10% or 12% over 5 to 15 days of continuous cell culture; 2%, 4%, 6%, 8%, 10%, 12%, 14%, 16%, 18% or 20% over 16 to 20 days of continuous cell culture; 2%, 4%, 6%, 8%, 10%, 12%, 14%, 16%, 18%, 20%, 22%, 24% over 21 to 30 days of continuous cell culture; 2%, 4%, 6%, 8%, 10%, 12%, 14%, 16%, 18%, 20%, 22%, 24%, 26%, 28% or 30% over 30 to 40 days of continuous cell culture; 2%, 4%, 6%, 8%, 10%, 12%, 14%, 16%, 18%, 20%, 22%, 24%, 26%, 28% or 30% over 41 to 45 days of continuous cell culture; 2%, 4%, 6%, 8%, 10%, 12%, 14%, 16%, 18%, 20%, 22%, 24%, 26%, 28% or 30% over 45 to 50 days of continuous cell culture; 2%, 4%, 6%, 8%, 10%, 12%, 14%, 16%, 18%, 20%, 22%, 24%, 26%, 28%, 30% or 35% over 45 to 50 days of continuous cell culture, 2%, 4%, 6%, 8%, 10%, 12%, 14%, 16%, 18%, 20%, 22%, 24%, 26%, 28% or 30% over 50 to 55 days of continuous cell culture; 2%, 4%, 6%, 8%, 10%, 12%, 14%, 16%, 18%, 20%, 22%, 24%, 26%, 28%, 30% or 35% over 50 to 55 days of continuous cell culture; 1%, 2%, 3%, 4%, 5%, 10%, 15%, 20%, 25%, 30%, 35% or 40% over 55 to 75 days of continuous cell culture; 1%, 2%, 3%, 4%, 5%, 6%, 10%, 15%, 20%, 25%, 30%, 35%, 40% or 45% over 75 to 100 days of continuous cell culture; 1%, 2%, 3%, 4%, 5%, 6%, 10%, 15%, 20%, 25%, 30%, 35%, 40% or 45% over 101 to 125 days of continuous cell culture; 1%, 2%, 3%, 4%, 5%, 6%, 10%, 15%, 20%, 25%, 30%, 35%, 40% or 45% over 126 to 150 days of continuous cell culture; 1%, 2%, 3%, 4%, 5%, 6%, 10%, 15%, 20%, 25%, 30%, 35%, 40% or 45% over 151 to 175 days of continuous cell culture; 1%, 2%, 3%, 4%, 5%, 6%, 10%, 15%, 20%, 25%, 30%, 35%, 40% or 45% over 176 to 200 days of continuous cell culture; or 1%, 2%, 3%, 4%, 5%, 6%, 10%, 15%, 20%, 25%, 30%, 35%, 40% or 45% over more than 200 days of continuous cell culture.

The nucleic acid encoding GC-C can be genomic DNA or cDNA. In some embodiments, the nucleic acid encoding the GC-C comprises one or more substitutions, mutations or deletions, as compared to a wild-type GC-C, that may or may not result in an amino acid substitution. In some embodiments, the nucleic acid is a fragment of the nucleic acid sequence provided. Such GC-Cs that are fragments or have such modifications retain at least one biological property of GC-C, e.g., its guanylyl cyclase activity, effect on cGMP levels, stimulation or reduction of water or chloride secretion, regulation of mucosal and/or epithelial fluid absorption or secretion, activation by guanylin, lymphoguanylin, uroguanylin, STa, linaclotide or SP-304, or inhibition by 5-(3-bromophenyl)-1,3-dimethyl-5,11-dihydro-1H-indeno[2′,1′:5,6]pyrido[2,3-d]pyrimidine-2,4,6-trione (BPIPP), or Tyrphostin A25 (AG82).

The invention encompasses cells and cell lines stably expressing a GC-C-coding nucleotide sequence that is at least about 85% identical to a GC-C-coding sequence disclosed herein. In some embodiments, the GC-C-encoding sequence identity is at least 85%, 90%, 95%, 96%, 97%, 98%, 99% or higher compared to a sequence provided herein. The invention also encompasses cells and cell lines containing a GC-C-coding nucleic acid that hybridizes under stringent conditions to a sequence provided herein.

In some embodiments, the cell or cell line comprises a GC-C-encoding nucleic acid sequence comprising a substitution compared to a sequence provided herein by at least one but less than 10, 20, 30, or 40 nucleotides, up to or equal to 1%, 5%, 10% or 20% of the nucleotide sequence; or a sequence substantially identical thereto (e.g., a sequence at least 85%, 90%, 95%, 96%, 97%, 98%, 99% or higher identical thereto, or that is capable of hybridizing under stringent conditions to the sequences disclosed). Such substitutions include single nucleotide polymorphisms (SNPs) and other allelic variations. In some embodiments, the cell or cell line comprises a GC-C-coding nucleic acid sequence comprising an insertion into or deletion from the sequences provided herein by less than 10, 20, 30, or 40 nucleotides up to or equal to 1%, 5%, 10% or 20% of the nucleotide sequence. The substitutions, insertions and deletions described herein may occur in any of the polynucleotides encoding GC-C in the cells or cell lines of the invention.

In some embodiments, where the nucleic acid substitution or modification results in an amino acid change, such as an amino acid substitution, the native amino acid may be replaced by a conservative or non-conservative substitution. In some embodiments, the sequence identity between the original and modified polypeptide sequence can differ by about 1%, 5%, 10% or 20% of the polypeptide sequence or from a sequence substantially identical thereto (e.g., a sequence at least 85%, 90%, 95%, 96%, 97%, 98%, 99% or higher identical thereto). Those of skill in the art will understand that a conservative amino acid substitution is one in which the amino acid side chains are similar in structure and/or chemical properties and the substitution should not substantially change the structural characteristics of the parent sequence. In embodiments comprising a nucleic acid comprising a mutation, the mutation may be a random mutation or a site-specific mutation.

Conservative modifications will produce GC-C having functional and chemical characteristics similar to those of the unmodified GC-C. A “conservative amino acid substitution” is one in which an amino acid residue is substituted by another amino acid residue having a side chain R group with similar chemical properties to the parent amino acid residue (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, Methods Mol. Biol. 243:307-31 (1994).

Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative amino acid substitution is any change having a positive value in the PAM250 log-likelihood matrix disclosed in Gonnet et al., Science 256:1443-45 (1992). A “moderately conservative” replacement is any change having a nonnegative value in the PAM250 log-likelihood matrix.

In some embodiments, a GC-C may be a chimeric protein comprising sequences from two or more species.

The invention further encompasses a collection or panel of cell lines comprising GC-C and at least one other receptor, a different guanylyl cyclase family member or a related protein. Non-limiting examples of guanylyl cyclase family members and related proteins include GC-A (SEQ ID NO: 4), GC-B (SEQ ID NO: 5), GC-D (SEQ ID NO: 6), GC-E (SEQ ID NO: 7), GC-F (SEQ ID NO: 8), GC-G (SEQ ID NO: 9) and NPR3 (SEQ ID NO: 10). In some embodiments, some of the cell lines in the collection may comprise the same protein (e.g., receptor). In some embodiments, some of the cell lines may comprise a control receptor outside the guanylyl cyclase family. The invention also encompasses a collection of cell lines comprising GC-C and mutant GC-Cs that are encoded by nucleic acids comprising at least one substitution, insertion or deletion compared to SEQ ID NO: 2. The substitution may be a single nucleotide polymorphism (SNP). In some embodiments, the mutant GC-Cs are allelic variants. The invention also encompasses cells or cell lines that express GC-C and one or more additional proteins, such as CFTR, CFTR mutants, cyclic nucleotide gated channels (e.g., CNGA2), GCAPs, cGKI, cGKII, 3′,5′-cyclic nucleotide phosphodiesterases (PDEs) and cGMP biosensors (see, e.g., National Institute of Neurological Disorders and Stroke, Grant Number 1R21NS059509-01, http://crisp.cit.nih.gov/).

In some embodiments, the GC-C-coding nucleic acid sequence further comprises a tag. Such tags may encode, for example, a HIS tag, a myc tag, a hemagglutinin (HA) tag, protein C, VSV-G, FLU, yellow fluorescent protein (YFP), green fluorescent protein (GFP), FLAG, BCCP, maltose binding protein tag, Nus-tag, Softag-1, Softag-2, Strep-tag, S-tag, thioredoxin, GST, V5, TAP or CBP. A tag may be used as a marker to determine GC-C expression levels, intracellular localization, protein-protein interactions, GC-C regulation, or GC-C function. Tags may also be used to purify or fractionate GC-C.

Host cells used to produce a cell or cell line of the invention may express GC-C in their native state. The host cell may be a primary, germ, or stem cell, including an embryonic stem cell. The host cell may also be an immortalized cell. Primary or immortalized host cells may be derived from mesoderm, ectoderm or endoderm layers of eukaryotic organisms. The host cell may be endothelial, epidermal, mesenchymal, neural, renal, hepatic, hematopoietic, or immune cells. For example, the host cells may be intestinal crypt or villi cells, Clara cells, colon cells, intestinal cells, goblet cells, enterochromafin cells, enteroendocrine cells. The host cells may be eukaryotic, prokaryotic, mammalian, human, primate, bovine, porcine, feline, rodent, marsupial, murine or other cells. The host cells may also be nonmammalian, such as yeast, insect, fungus, plant, lower eukaryotes and prokaryotes. Such host cells may provide backgrounds that are more divergent for testing GC-C modulators with a greater likelihood for the absence of expression products provided by the cell that may interact with the target. In preferred embodiments, the host cell is a mammalian cell. Examples of host cells that may be used to produce a cell or cell line of the invention include but are not limited to: 293T cells, established neuronal cell lines, pheochromocytomas, neuroblastomas fibroblasts, rhabdomyosarcomas, dorsal root ganglion cells, NS0 cells, CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), L-cells, HEK-293 (ATCC CRL1573) and PC12 (ATCC CRL-1721), HEK293T (ATCC CRL-11268), RBL (ATCC CRL-1378), SH-SY5Y (ATCC CRL-2266), MDCK (ATCC CCL-34), SJ-RH30 (ATCC CRL-2061), HepG2 (ATCC HB-8065), ND7/23 (ECACC 92090903), CHO (ECACC 85050302), Vero (ATCC CCL 81), Caco-2 (ATCC HTB 37), K562 (ATCC CCL 243), Jurkat (ATCC TIB-152), Per.C6 (Crucell, Leiden, The Netherlands), Huvec (ATCC Human Primary PCS 100-010, Mouse CRL 2514, CRL 2515, CRL 2516), HuH-7D12 (ECACC 01042712), 293 (ATCC CRL 10852), A549 (ATCC CCL 185), IMR-90 (ATCC CCL 186), MCF-7 (ATC HTB-22), U-2 OS (ATCC HTB-96), T84 (ATCC CCL 248), or any established cell line (polarized or nonpolarized) or any cell line available from repositories such as the American Type Culture Collection (ATCC, 10801 University Blvd. Manassas, Va. 20110-2209 USA) or European Collection of Cell Cultures (ECACC, Salisbury Wiltshire SP4 0JG England). In some embodiments, the host cell is a 293T cell. Host cells used to produce a cell or cell line of the invention may be in suspension. For example, the host cells may be adherent cells adapted to suspension.

In one embodiment, the host cell is an embryonic stem cell that is then used as the basis for the generation of transgenic animals. Embryonic stem cells stably expressing GC-C, and preferably a functional introduced GC-C, may be implanted into organisms directly, or their nuclei may be transferred into other recipient cells and these may then be implanted in vivo for studying growth and development. The embryonic stem cells also may be used to create transgenic animals.

As will be appreciated by those of skill in the art, any vector that is suitable for use with the host cell may be used to introduce a nucleic acid encoding GC-C into the host cell. Examples of vectors that may be used to introduce GC-C-encoding nucleic acids into host cells include but are not limited to plasmids, viruses, including retroviruses and lentiviruses, cosmids, artificial chromosomes and may include for example, pFN11A (BIND) Flexi®, pGL4.31, pFC14A (HaloTag® 7) CMV Flexi®, pFC14K (HaloTag® 7) CMV Flexi®, pFN24A (HaloTag® 7) CMVd3 Flexi®, pFN24K (HaloTag® 7) CMVd3 Flexi®, HaloTag™ pHT2, pACT, pAdVAntage™, pALTER®-MAX, pBIND, pCAT®3-Basic, pCAT®3-Control, pCAT®3-Enhancer, pCAT®3-Promoter, pCI, pCMVTNT™, pG5luc, pSI, pTARGET™, pTNT™, pF12A RM Flexi®, pF12K RM Flexi®, pReg neo, pYES2/GS, pAd/CMV/V5-DEST Gateway® Vector, pAd/PL-DEST™ Gateway® Vector, Gateway® pDEST™27 Vector, Gateway® pEF-DEST51 Vector, Gateway® pcDNA™-DEST47 vector, pCMV/Bsd Vector, pEF6/His A, B, & C, pcDNA™6.2-DEST, pLenti6/TR, pLP-AcGFP1-C, pLPS-AcGFP1-N, pLP-IRESneo, pLP-TRE2, pLP-RevTRE, pLP-LNCX, pLP-CMV-HA, pLP-CMV-Myc, pLP-RetroQ, pLP-CMVneo, pCMV-Script, pcDNA3.1 Hygro, pcDNA3.1neo, pcDNA3.1puro, pSV2neo, pIRES puro, and pSV2 zeo. In some embodiments, the vectors comprise expression control sequences such as constitutive or conditional promoters. One of ordinary skill in the art will be able to select such sequences. For example, suitable promoters include but are not limited to CMV, TK, SV40 and EF-1α. In some embodiments, the promoters are inducible, temperature regulated, tissue specific, repressible, heat-shock, developmental, cell lineage specific, eukaryotic, prokaryotic or temporal promoters or a combination or recombination of unmodified or mutagenized, randomized, shuffled sequences of any one or more of the above. In other embodiments, GC-C is expressed by gene activation or when a gene encoding GC-C is episomal. Nucleic acids encoding GC-C are preferably constitutively expressed.

In some embodiments, the vector encoding GC-C lacks a selectable marker or drug resistance gene. In other embodiments, the vector optionally comprises a nucleic acid encoding a selectable marker such as a protein that confers drug or antibiotic resistance. Suitable markers will be well-known to those of skill in the art and include but are not limited to genes conferring resistance to any one of the following: Neomycin/G418, Puromycin, hygromycin, Zeocin, methotrexate and blasticidin. Although drug selection (or selection using any other suitable selection marker) is not a required step, it may be used to enrich the transfected cell population for stably transfected cells, provided that the transfected constructs are designed to confer drug resistance. If subsequent selection of cells expressing GC-C is accomplished using signaling probes, selection too soon following transfection can result in some positive cells that may only be transiently and not stably transfected. However, this can be minimized by allowing sufficient cell passage allowing for dilution of transient expression in transfected cells.

In some embodiments, the vector comprises a nucleic acid sequence encoding an RNA tag sequence. “Tag sequence” refers to a nucleic acid sequence that is an expressed RNA or portion of an RNA that is to be detected by a signaling probe. Signaling probes may detect a variety of RNA sequences. Any of these RNAs may be used as tags. Signaling probes may be directed against the RNA tag by designing the probes to include a portion that is complementary to the sequence of the tag. The tag sequence may be a 3′ untranslated region of the plasmid that is co-transcribed and comprises a target sequence for signaling probe binding. The RNA encoding the gene of interest may include the tag sequence or the tag sequence may be located within a 5′-untranslated region or 3′-untranslated region. In some embodiments, the tag is not with the RNA encoding the gene of interest. The tag sequence can be in frame with the protein-coding portion of the message of the gene or out of frame with it, depending on whether one wishes to tag the protein produced. Thus, the tag sequence does not have to be translated for detection by the signaling probe. The tag sequences may comprise multiple target sequences that are the same or different, wherein one signaling probe hybridizes to each target sequence. The tag sequences may encode an RNA having secondary structure. The structure may be a three-arm junction structure. Examples of tag sequences that may be used in the invention, and to which signaling probes may be prepared, include but are not limited to the RNA transcript of epitope tags such as, for example, a HIS tag, a myc tag, a hemagglutinin (HA) tag, protein C, VSV-G, FLU, yellow fluorescent protein (YFP), green fluorescent protein (GFP), FLAG, BCCP, maltose binding protein tag, Nus-tag, Softag-1, Softag-2, Strep-tag, S-tag, thioredoxin, GST, V5, TAP or CBP. As described herein, one of ordinary skill in the art could create his or her own RNA tag sequences.

In another aspect of the invention, cells and cell lines of the invention have enhanced stability as compared to cells and cell lines produced by conventional methods. To identify stable expression, a cell or cell line's expression of GC-C is measured over a time course and the expression levels are compared. Stable cell lines will continue expressing GC-C throughout the time course. In some aspects of the invention, the time course may be for at least one week, two weeks, three weeks, etc., or at least one month, or at least two, three, four, five, six, seven, eight or nine months, or any length of time in between. Isolated cells and cell lines can be further characterized, such as by qRT-PCR and single end-point RT-PCR to determine the absolute amounts and relative amounts of GC-C being expressed. In some embodiments, stable expression is measured by comparing the results of functional assays over a time course. The measurement of stability based on functional assay provides the benefit of identifying clones that not only stably express the mRNA of the gene of interest, but also stably produce and properly process (e.g., post-translational modification, subunit assembly, and localization within the cell) the protein encoded by the gene of interest that functions appropriately.

Cells and cell lines of the invention have the further advantageous property of providing assays with high reproducibility as evidenced by their Z′ factor. See Zhang J H, Chung T D, Oldenburg K R, “A Simple Statistical Parameter for Use in Evaluation and Validation of High Throughput Screening Assays.” J. Biomol. Screen. 1999; 4(2):67-73. Z′ values pertain to the quality of a cell or cell line because it reflects the degree to which a cell or cell line will respond consistently to modulators. Z′ is a statistical calculation that takes into account the signal-to-noise range and signal variability (i.e., from well to well) of the functional response to a reference compound across a multiwell plate. Z′ is calculated using data obtained from multiple wells with a positive control and multiple wells with a negative control. The ratio of their combined standard deviations multiplied by three to the difference in their mean values is subtracted from one to give the Z′ factor, according the equation below:

Z′ factor=1−((3σ_{positive control}+3σ_{negative control})/(μ_{positive control}−_{negative control}))

The theoretical maximum Z′ factor is 1.0, which would indicate an ideal assay with no variability and limitless dynamic range. As used herein, a “high Z′” refers to a Z′ factor of Z′ of at least 0.6, at least 0.7, at least 0.75 or at least 0.8, or any decimal in between 0.6 and 1.0. A score less than 0 is undesirable because it indicates that there is overlap between positive and negative controls. In the industry, for simple cell-based assays, Z′ scores up to 0.3 are considered marginal scores, Z′ scores between 0.3 and 0.5 are considered acceptable, and Z′ scores above 0.5 are considered excellent. Cell-free or biochemical assays may approach higher Z′ scores, but Z′ scores for cell-based systems tend to be lower because cell-based systems are complex.

As those of ordinary skill in the art will recognize, historically, cell-based assays using cells expressing a single chain protein do not typically achieve a Z′ higher than 0.5 to 0.6. Such cells would not be reliable to use in an assay because the results are not reproducible. Cells and cell lines of the invention, on the other hand, have high Z′ values and advantageously produce consistent results in assays. GC-C cells and cell lines of the invention provide the basis for high throughput screening (HTS) compatible assays because they generally have Z′ factors at least 0.7. In some aspects of the invention, the cells and cell lines result in Z′ of at least 0.3, at least 0.4, at least 0.5, at least 0.6, at least 0.7, or at least 0.8. In other aspects of the invention, the cells and cell lines of the invention result in a Z′ of at least 0.7, at least 0.75 or at least 0.8 maintained for multiple passages, e.g., between 5-20 passages, including any integer in between 5 and 20. In some aspects of the invention, the cells and cell lines result in a Z′ of at least 0.7, at least 0.75 or at least 0.8 maintained for 1, 2, 3, 4 or 5 weeks or 2, 3, 4, 5, 6, 7, 8 or 9 months, including any period of time in between.

In some embodiments, the cells and cell lines of the invention express GC-C with “physiologically relevant” activity. As used herein, physiological relevance refers to a property of a cell or cell line expressing GC-C whereby the GC-C possesses guanylyl cyclase activity, affects cGMP levels, affects water or chloride secretion or regulates mucosal and/or epithelial fluid absorption or secretion in the same way as a naturally occurring GC-C and responds to modulators in substantially the same way that naturally occurring GC-C is modulated by the same compounds. GC-C cells and cell lines of this invention preferably demonstrate comparable function to cells that normally express native GC-C in a suitable assay, such as a competitive ELISA for cGMP levels after the GC-C expressing cells are treated with a modulator, a radioimmunoassay or a protein kinase assay. Such comparisons are used to determine a cell or cell line's physiological relevance.

In some embodiments, the cells and cell lines of the invention have increased sensitivity to modulators of GC-C. Cells and cell lines of the invention respond to modulators and produce cGMP, possess guanylyl cyclase activity, stimulate water or chloride secretion or regulate mucosal and/or epithelial fluid absorption or secretion with physiological range EC₅₀ or IC₅₀ values for GC-C. As used herein, EC₅₀ refers to the concentration of a compound or substance required to induce a half-maximal activating response in the cell or cell line. As used herein, IC₅₀ refers to the concentration of a compound or substance required to induce a half-maximal inhibitory response in the cell or cell line. EC₅₀ and IC₅₀ values may be determined using techniques that are well-known in the art, for example, a dose-response curve that correlates the concentration of a compound or substance to the response of the GC-C-expressing cell line. For example, the EC50 for guanylin in a cell line of the invention is about 1.1 nM.

A further advantageous property of the GC-C cells and cell lines of the inventions, flowing from the physiologically relevant function of the GC-C is that modulators identified in initial screening are functional in secondary functional assays, e.g., rabbit intestinal loop assay, animal studies measuring fecal output, Ussing chamber assays and electrophysiology to assess GC-C function. As those of ordinary skill in the art will recognize, compounds identified in initial screening assays typically must be modified, such as by combinatorial chemistry, medicinal chemistry or synthetic chemistry, for their derivatives or analogs to be functional in secondary functional assays. However, due to the high physiological relevance of the present GC-C cells and cell lines, many compounds identified therewith are functional without “coarse” tuning.

In some embodiments, properties of the cells and cell lines of the invention, such as stability, physiological relevance, reproducibility in an assay (Z′), or physiological EC₅₀ or IC₅₀ values, are achievable under specific culture conditions. In some embodiments, the culture conditions are standardized and rigorously maintained without variation, for example, by automation. Culture conditions may include any suitable conditions under which the cells or cell lines are grown and may include those known in the art. A variety of culture conditions may result in advantageous biological properties for GC-C, or its mutants or allelic variants.

In other embodiments, the cells and cell lines of the invention with desired properties, such as stability, physiological relevance, reproducibility in an assay (Z′), or physiological EC₅₀ or IC₅₀ values, can be obtained within one month or less. For example, the cells or cell lines may be obtained within 2, 3, 4, 5, or 6 days, or within 1, 2, 3 or 4 weeks, or any length of time in between.

One aspect of the invention provides a collection of clonal cells and cell lines, each expressing a GC-C. The collection may include, for example, cells or cell lines expressing combinations or full length or fragments of GC-C. The collection may also include, for example, other guanylyl cyclase (GC) family members or related proteins.

When collections or panels of cells or cell lines are produced, e.g., for drug screening, the cells or cell lines in the collection or panel may be derived from the same host cells and may be matched such that they are the same (including substantially the same) with regard to one or more selective physiological properties. The “same physiological property” in this context means that the selected physiological property is similar enough amongst the members in the collection or panel such that the cell collection or panel can produce reliable results in drug screening assays; for example, variations in readouts in a drug screening assay will be due to, e.g., the different biological activities of test compounds on cells expressing different forms of guanylyl cyclase (GC) proteins, rather than due to inherent variations in the cells. For example, the cells or cell lines may be matched to have the same growth rate, i.e., growth rates with no more than one, two, three, four, or five hour difference amongst the members of the cell collection or panel. This may be achieved by, for example, binning cells by their growth rate into five, six, seven, eight, nine, or ten groups, and creating a panel using cells from the same binned group. Methods of determining cell growth rate are well known in the art. The cells or cell lines in a panel also can be matched to have the same Z′ factor (e.g., Z′ factors that do not differ by more than 0.1), GC expression level (e.g., GC expression levels that do not differ by more than 5%, 10%, 15%, 20%, 25%, or 30%), adherence to tissue culture surfaces, and the like. Matched cells and cell lines can be grown under identical conditions, achieved by, e.g., automated parallel processing, to maintain the selected physiological property.

Matched cell panels of the invention can be used to, for example, identify modulators with defined activity (e.g., agonist or antagonist) on GC proteins; to profile compound activity across different forms of GC proteins; to identify modulators active on just one form of GC; and to identify modulators active on just a subset of GCs. The matched cell panels of the invention allow high throughput screening. Screenings that used to take months to accomplish can now be accomplished within weeks.

To make cells and cell lines of the invention, one can use, for example, the technology described in U.S. Pat. No. 6,692,965 and International Patent Publication WO/2005/079462. Both of these documents are incorporated herein by reference in their entirety for all purposes. This technology provides real-time assessment of millions of cells such that any desired number of clones (from hundreds to thousands of clones) may be selected. Using cell sorting techniques, such as flow cytometric cell sorting (e.g., with a FACS machine), magnetic cell sorting (e.g., with a MACS machine) or fluorescence plate readers, including those compatible with high throughput screening, one cell per well may be automatically deposited with high statistical confidence in a culture vessel (such as a 96-well culture plate). The speed and automation of the technology allows multigene cell lines to be readily isolated.

Using the technology, the RNA sequence for GC-C may be detected using a signaling probe, also referred to as a molecular beacon or fluorogenic probe. In some embodiments, the molecular beacon recognizes a target tag sequence as described above. In another embodiment, the molecular beacon recognizes a sequence within the GC-C itself. Signaling probes may be directed against the RNA tag or GC-C sequence by designing the probes to include a portion that is complementary to the RNA sequence of the tag or GC-C, respectively.

Nucleic acids comprising a sequence encoding GC-C, or the sequence of GC-C and a tag sequence, and optionally a nucleic acid encoding a selectable marker may be introduced into selected host cells by well known methods. The methods include but not limited to transfection, viral delivery, protein or peptide mediated insertion, co-precipitation methods, lipid based delivery reagents (lipofection), cytofection, lipopolyamine delivery, dendrimer delivery reagents, electroporation or mechanical delivery. Examples of transfection reagents are GENEPORTER, GENEPORTER2, LIPOFECTAMINE™, LIPOFECTAMINE™ 2000, FUGENE® 6, FUGENE® HD, TFX™-10, TFX™-20, TFX™-50, OLIGOFECTAMINE™, TRANSFAST, TRANSFECTAM, GENESHUTTLE, TROJENE, GENESILENCER, X-TREMEGENE, PERFECTIN, CYTOFECTIN, SIPORT, UNIFECTOR, SIFECTOR, TRANSIT-LT1, TRANSIT-LT2, TRANSIT-EXPRESS, IFECT, RNAI SHUTTLE, METAFECTENE, LYOVEC, LIPOTAXI, GENEERASER, GENEJUICE, CYTOPURE, JETSI, JETPEI, MEGAFECTIN, POLYFECT, TRANSMESSANGER, RNAiFECT, SUPERFECT, EFFECTENE, TF-PEI-KIT, CLONFECTIN, METAFECTINE, DOTAP/DOPE and FECTURIN™.

Following introduction of the GC-C coding sequences into host cells and optional subsequent drug selection, molecular beacons (e.g., fluorogenic probes) are introduced into the cells and cell sorting is used to isolate cells positive for their signals. Multiple rounds of sorting may be carried out, if desired. In one embodiment, the flow cytometric cell sorter is a FACS machine. MACS (magnetic cell sorting) or laser ablation of negative cells using laser-enabled analysis and processing can also be used. According to this method, cells expressing GC-C are detected and recovered. The expression level in a cell or cell line also may decrease over time due to epigenetic events such as DNA methylation and gene silencing and loss of transgene copies. These variations can be attributed to a variety of factors, for example, the copy number of the transgene taken up by the cell, the site of genomic integration of the transgene, and the integrity of the transgene following genomic integration. The GC-C sequences may be integrated at different locations of the genome in the cell. The expression level of the introduced genes encoding the GC-C may vary based upon integration site. The skilled worker will recognize that sorting can be gated for any desired expression level.

Signaling probes useful in this invention are known in the art and generally are oligonucleotides comprising a sequence complementary to a target sequence and a signal emitting system so arranged that no signal is emitted when the probe is not bound to the target sequence and a signal is emitted when the probe binds to the target sequence. By way of non-limiting illustration, the signaling probe may comprise a fluorophore and a quencher positioned in the probe so that the quencher and fluorophore are brought together in the unbound probe. Upon binding between the probe and the target sequence, the quencher and fluorophore separate, resulting in emission of signal. International Patent Publication WO/2005/079462, for example, describes a number of signaling probes that may be used in the production of the cells and cell lines of this invention.

Nucleic acids encoding signaling probes may be introduced into the selected host cell by any of numerous means that will be well-known to those of skill in the art, including but not limited to transfection, co-precipitation methods, lipid based delivery reagents (lipofection), cytofection, lipopolyamine delivery, dendrimer delivery reagents, electroporation or mechanical delivery. Examples of transfection reagents are GENEPORTER, GENEPORTER2, LIPOFECTAMINE™, LIPOFECTAMINE™ 2000, FUGENE® 6, FUGENE® HD, TFX™-10, TFX™-20, TFX™-50, OLIGOFECTAMINE™, TRANSFAST, TRANSFECTAM, GENESHUTTLE, TROJENE, GENESILENCER, X-TREMEGENE, PERFECTIN, CYTOFECTIN, SIPORT, UNIFECTOR, SIFECTOR, TRANSIT-LT1, TRANSIT-LT2, TRANSIT-EXPRESS, IFECT, RNAI SHUTTLE, METAFECTENE, LYOVEC, LIPOTAXI, GENEERASER, GENEJUICE, CYTOPURE, JETSI, JETPEI, MEGAFECTIN, POLYFECT, TRANSMESSANGER, RNAiFECT, SUPERFECT, EFFECTENE, TF-PEI-KIT, CLONFECTIN, METAFECTINE, DOTAP/DOPE and FECTURIN™.

In one embodiment, the signaling probes are designed to be complementary to either a portion of the RNA encoding GC-C or to portions of its 5′ or 3′ untranslated regions. If the signaling probe designed to recognize a messenger RNA of interest is able to detect spurious endogenously existing target sequences, the proportion of these in comparison to the proportion of the sequence of interest produced by transfected cells is such that the sorter is able to discriminate the two cell types.

The expression level of GC-C may vary from cell or cell line to cell or cell line. The expression level in a cell or cell line also may decrease over time due to epigenetic events such as DNA methylation and gene silencing and loss of transgene copies. These variations can be attributed to a variety of factors, for example, the copy number of the transgene taken up by the cell, the site of genomic integration of the transgene, and the integrity of the transgene following genomic integration. One may use FACS or other cell sorting methods (i.e., MACS) to evaluate expression levels. Additional rounds of introducing signaling probes may be used, for example, to determine if and to what extent the cells remain positive over time for any one or more of the RNAs for which they were originally isolated.

In another embodiment of the invention, adherent cells can be adapted to suspension before or after cell sorting and isolating single cells. In other embodiments, isolated cells may be grown individually or pooled to give rise to populations of cells. Individual or multiple cell lines may also be grown separately or pooled. If a pool of cell lines is producing a desired activity or has a desired property, it can be further fractionated until the cell line or set of cell lines having this effect is identified. Pooling cells or cell lines may make it easier to maintain large numbers of cell lines without the requirements for maintaining each separately. Thus, a pool of cells or cell lines may be enriched for positive cells. An enriched pool may have at least 50%, at least 60%, at least 70%, at least 80% or at least 90%, or 100% are positive for the desired property or activity.

In a further aspect, the invention provides a method for producing the GC-C-expressing cells and cell lines of the invention. In one embodiment, the method comprises the steps of:
a) providing a plurality of cells that express mRNA encoding the GC-C;
b) dispersing cells individually into individual culture vessels, thereby providing a plurality of separate cell cultures
c) culturing the cells under a set of desired culture conditions using automated cell culture methods characterized in that the conditions are substantially identical for each of the separate cell cultures, during which culturing the number of cells in each separate cell culture is normalized, and wherein the separate cultures are passaged on the same schedule;
d) assaying the separate cell cultures for at least one desired characteristic of the GC-C at least twice; and
e) identifying a separate cell culture that expresses the GC-C at a consistent level in both assays.

According to the method, the cells are cultured under a desired set of culture conditions. The conditions can be any desired conditions. Those of skill in the art will understand what parameters are comprised within a set of culture conditions. For example, culture conditions include but are not limited to: the media (Base media (DMEM, MEM, RPMI, serum-free, with serum, fully chemically defined, without animal-derived components), mono and divalent ion (sodium, potassium, calcium, magnesium) concentration, additional components added (amino acids, antibiotics, glutamine, glucose or other carbon source, HEPES, channel blockers, modulators of other targets, vitamins, trace elements, heavy metals, co-factors, growth factors, anti-apoptosis reagents), fresh or conditioned media, with HEPES, pH, depleted of certain nutrients or limiting (amino acid, carbon source)), level of confluency at which cells are allowed to attain before split/passage, feeder layers of cells, or gamma-irradiated cells, CO₂, a three gas system (oxygen, nitrogen, carbon dioxide), humidity, temperature, still or on a shaker, and the like, which will be well known to those of skill in the art.

The cell culture conditions may be chosen for convenience or for a particular desired use of the cells. Advantageously, the invention provides cells and cell lines that are optimally suited for a particular desired use. That is, in embodiments of the invention in which cells are cultured under conditions for a particular desired use, cells are selected that have desired characteristics under the condition for the desired use. By way of illustration, if cells will be used in assays in plates where it is desired that the cells are adherent, cells that display adherence under the conditions of the assay may be selected. Similarly, if the cells will be used for protein production, cells may be cultured under conditions appropriate for protein production and selected for advantageous properties for this use.

In some embodiments, the method comprises the additional step of measuring the growth rates of the separate cell cultures. Growth rates may be determined using any of a variety of techniques means that will be well known to the skilled worker. Such techniques include but are not limited to measuring ATP, cell confluency, light scattering, optical density (e.g., OD 260 for DNA). Preferably growth rates are determined using means that minimize the amount of time that the cultures spend outside the selected culture conditions.

In some embodiments, cell confluency is measured and growth rates are calculated from the confluency values. In some embodiments, cells are dispersed and clumps removed prior to measuring cell confluency for improved accuracy. Means for monodispersing cells are well-known and can be achieved, for example, by addition of a dispersing reagent to a culture to be measured. Dispersing agents are well-known and readily available, and include but are not limited to enzymatic dispering agents, such as trypsin, and EDTA-based dispersing agents. Growth rates can be calculated from confluency date using commercially available software for that purpose such as HAMILTON VECTOR. Automated confluency measurement, such as using an automated microscopic plate reader is particularly useful. Plate readers that measure confluency are commercially available and include but are not limited to the CLONE SELECT IMAGER (Genetix). Typically, at least 2 measurements of cell confluency are made before calculating a growth rate. The number of confluency values used to determine growth rate can be any number that is convenient or suitable for the culture. For example, confluency can be measured multiple times over e.g., a week, 2 weeks, 3 weeks or any length of time and at any frequency desired.

When the growth rates are known, according to the method, the plurality of separate cell cultures are divided into groups by similarity of growth rates. By grouping cultures into growth rate bins, one can manipulate the cultures in the group together, thereby providing another level of standardization that reduces variation between cultures. For example, the cultures in a bin can be passaged at the same time, treated with a desired reagent at the same time, etc. Further, functional assay results are typically dependent on cell density in an assay well. A true comparison of individual clones is only accomplished by having them plated and assayed at the same density. Grouping into specific growth rate cohorts enables the plating of clones at a specific density that allows them to be functionally characterized in a high throughput format.

The range of growth rates in each group can be any convenient range. It is particularly advantageous to select a range of growth rates that permits the cells to be passaged at the same time and avoid frequent renormalization of cell numbers. Growth rate groups can include a very narrow range for a tight grouping, for example, average doubling times within an hour of each other. But according to the method, the range can be up to 2 hours, up to 3 hours, up to 4 hours, up to 5 hours or up to 10 hours of each other or even broader ranges. The need for renormalization arises when the growth rates in a bin are not the same so that the number of cells in some cultures increases faster than others. To maintain substantially identical conditions for all cultures in a bin, it is necessary to periodically remove cells to renormalize the numbers across the bin. The more disparate the growth rates, the more frequently renormalization is needed.

In step d) the cells and cell lines may be tested for and selected for any physiological property including but not limited to: a change in a cellular process encoded by the genome; a change in a cellular process regulated by the genome; a change in a pattern of chromosomal activity; a change in a pattern of chromosomal silencing; a change in a pattern of gene silencing; a change in a pattern or in the efficiency of gene activation; a change in a pattern or in the efficiency of gene expression; a change in a pattern or in the efficiency of RNA expression; a change in a pattern or in the efficiency of RNAi expression; a change in a pattern or in the efficiency of RNA processing; a change in a pattern or in the efficiency of RNA transport; a change in a pattern or in the efficiency of protein translation; a change in a pattern or in the efficiency of protein folding; a change in a pattern or in the efficiency of protein assembly; a change in a pattern or in the efficiency of protein modification; a change in a pattern or in the efficiency of protein transport; a change in a pattern or in the efficiency of transporting a membrane protein to a cell surface change in growth rate; a change in cell size; a change in cell shape; a change in cell morphology; a change in % RNA content; a change in % protein content; a change in % water content; a change in % lipid content; a change in ribosome content; a change in mitochondrial content; a change in ER mass; a change in plasma membrane surface area; a change in cell volume; a change in lipid composition of plasma membrane; a change in lipid composition of nuclear envelope; a change in protein composition of plasma membrane; a change in protein; composition of nuclear envelope; a change in number of secretory vesicles; a change in number of lysosomes; a change in number of vacuoles; a change in the capacity or potential of a cell for: protein production, protein secretion, protein folding, protein assembly, protein modification, enzymatic modification of protein, protein glycosylation, protein phosphorylation, protein dephosphorylation, metabolite biosynthesis, lipid biosynthesis, DNA synthesis, RNA synthesis, protein synthesis, nutrient absorption, cell growth, mitosis, meiosis, cell division, to de-differentiate, to transform into a stem cell, to transform into a pluripotent cell, to transform into a omnipotent cell, to transform into a stem cell type of any organ (i.e., liver, lung, skin, muscle, pancreas, brain, testis, ovary, blood, immune system, nervous system, bone, cardiovascular system, central nervous system, gastro-intestinal tract, stomach, thyroid, tongue, gall bladder, kidney, nose, eye, nail, hair, taste bud), to transform into a differentiated any cell type (i.e. muscle, heart muscle, neuron, skin, pancreatic, blood, immune, red blood cell, white blood cell, killer T-cell, enteroendocrine cell, taste, secretory cell, kidney, epithelial cell, endothelial cell, also including any of the animal or human cell types already listed that can be used for introduction of nucleic acid sequences), to uptake DNA, to uptake small molecules, to uptake fluorogenic probes, to uptake RNA, to adhere to solid surface, to adapt to serum-free conditions, to adapt to serum-free suspension conditions, to adapt to scaled-up cell culture, for use for large scale cell culture, for use in drug discovery, for use in high throughput screening, for use in a functional cell based assay, for use in membrane potential assays, for use in reporter cell based assays, for use in ELISA studies, for use in in vitro assays, for use in in vivo applications, for use in secondary testing, for use in compound testing, for use in a binding assay, for use in panning assay, for use in an antibody panning assay, for use in imaging assays, for use in microscopic imaging assays, for use in multiwell plates, for adaptation to automated cell culture, for adaptation to miniaturized automated cell culture, for adaptation to large-scale automated cell culture, for adaptation to cell culture in multiwell plates (6, 12, 24, 48, 96, 384, 1536 or higher density), for use in cell chips, for use on slides, for use on glass slides, for microarray on slides or glass slides, for immunofluorescence studies, for use in protein purification, or for use in biologics production. Those of skill in the art will readily recognize suitable tests for any of the above-listed properties.

Tests that may be used to characterize cells and cell lines of the invention and/or matched panels of the invention include but are not limited to: amino acid analysis, DNA sequencing, protein sequencing, NMR, a test for protein transport, a test for nucleocytoplasmic transport, a test for subcellular localization of proteins, a test for subcellular localization of nucleic acids, microscopic analysis, submicroscopic analysis, fluorescence microscopy, electron microscopy, confocal microscopy, laser ablation technology, cell counting and dialysis. The skilled worker would understand how to use any of the above-listed tests.

According to the method, cells may be cultured in any cell culture format so long as the cells or cell lines are dispersed in individual cultures prior to the step of measuring growth rates. For example, for convenience, cells may be initially pooled for culture under the desired conditions and then individual cells separated one cell per well or vessel. Cells may be cultured in multi-well tissue culture plates with any convenient number of wells. Such plates are readily commercially available and will be well knows to a person of skill in the art. In some cases, cells may preferably be cultured in vials or in any other convenient format, the various formats will be known to the skilled worker and are readily commercially available.

In embodiments comprising the step of measuring growth rate, prior to measuring growth rates, the cells are cultured for a sufficient length of time for them to acclimate to the culture conditions. As will be appreciated by the skilled worker, the length of time will vary depending on a number of factors such as the cell type, the chosen conditions, the culture format and may be any amount of time from one day to a few days, a week or more.

Preferably, each individual culture in the plurality of separate cell cultures is maintained under substantially identical conditions as discussed below, including a standardized maintenance schedule. Another advantageous feature of the method is that large numbers of individual cultures can be maintained simultaneously, so that a cell with a desired set of traits may be identified even if extremely rare. For those and other reasons, according to the invention, the plurality of separate cell cultures are cultured using automated cell culture methods so that the conditions are substantially identical for each well. Automated cell culture prevents the unavoidable variability inherent to manual cell culture.

Any automated cell culture system may be used in the method of the invention. A number of automated cell culture systems are commercially available and will be well-known to the skilled worker. In some embodiments, the automated system is a robotic system. Preferably, the system includes independently moving channels, a multichannel head (for instance a 96-tip head) and a gripper or cherry-picking arm and a HEPA filtration device to maintain sterility during the procedure. The number of channels in the pipettor should be suitable for the format of the culture. Convenient pipettors have, e.g., 96 or 384 channels. Such systems are known and are commercially available. For example, a MICROLAB STAR™ instrument (Hamilton) may be used in the method of the invention. The automated system should be able to perform a variety of desired cell culture tasks. Such tasks will be known by a person of skill in the art. They include but are not limited to: removing media, replacing media, adding reagents, cell washing, removing wash solution, adding a dispersing agent, removing cells from a culture vessel, adding cells to a culture vessel and the like.

The production of a GC-C-expressing cell or cell line of the invention may include any number of separate cell cultures. However, the advantages provided by the method increase as the number of cells increases. There is no theoretical upper limit to the number of cells or separate cell cultures that can be utilized in the method. According to the invention, the number of separate cell cultures can be two or more but more advantageously is at least 3, 4, 5, 6, 7, 8, 9, 10 or more separate cell cultures, for example, at least 12, at least 15, at least 20, at least 24, at least 25, at least 30, at least 35, at least 40, at least 45, at least 48, at least 50, at least 75, at least 96, at least 100, at least 200, at least 300, at least 384, at least 400, at least 500, at least 1000, at least 10,000, at least 100,000, at least 500,000 or more.

A further advantageous property of the GC-C cells and cell lines of the invention is that they stably express GC-C in the absence of selective pressure. Selection pressure is applied in cell culture to select cells with desired sequences or traits, and is usually achieved by linking the expression of a polypeptide of interest with the expression of a selection marker that imparts to the cells resistance to a corresponding selective agent or pressure. Antibiotic selection includes, without limitation, the use of antibiotics (e.g., puromycin, neomycin, G418, hygromycin, bleomycin and the like). Non-antibiotic selection includes, without limitation, the use of nutrient deprivation, exposure to selective temperatures, exposure to mutagenic conditions and expression of fluorescent markers where the selection marker may be, e.g., glutamine synthetase, dihydrofolate reductase (DHFR), oabain, thymidine kinase (TK), hypoxanthine guanine phosphororibosyltransferase (HGPRT) or a fluorescent protein such as GFP. Thus, in some embodiments, cells and cell lines of the invention are maintained in culture without any selective pressure. In further embodiments, cells and cell lines are maintained without any antibiotics. As used herein, cell maintenance refers to culturing cells after they have been selected as described above for their GC-C expression. Maintenance does not refer to the optional step of growing cells in a selective drug (e.g., an antibiotic) prior to cell sorting where drug resistance marker(s) introduced into the cells allow enrichment of stable transfectants in a mixed population.

Drug-free cell maintenance provides a number of advantages. For examples, drug-resistant cells do not always express the co-transfected transgene of interest at adequate levels, because the selection relies on survival of the cells that have taken up the drug resistant gene, with or without the transgene. Further, selective drugs are often mutagenic or otherwise interfere with the physiology of the cells, leading to skewed results in cell-based assays. For example, selective drugs may decrease susceptibility to apoptosis (Robinson et al., Biochemistry, 36(37):11169-11178 (1997)), increase DNA repair and drug metabolism (Deffie et al., Cancer Res. 48(13):3595-3602 (1988)), increase cellular pH (Thiebaut et al., J Histochem Cytochem. 38(5):685-690 (1990); Roepe et al., Biochemistry. 32(41):11042-11056 (1993); Simon et al., Proc Natl Acad Sci USA. 91(3):1128-1132 (1994)), decrease lysosomal and endosomal pH (Schindler et al., Biochemistry. 35(9):2811-2817 (1996); Altan et al., J Exp Med. 187(10):1583-1598 (1998)), decrease plasma membrane potential (Roepe et al., Biochemistry. 32(41):11042-11056 (1993)), increase plasma membrane conductance to chloride (Gill et al., Cell. 71(1):23-32 (1992)) and ATP (Abraham et al., Proc Natl Acad Sci USA. 90(1):312-316 (1993)), and increase rates of vesicle transport (Altan et al., Proc Natl Acad Sci USA. 96(8):4432-4437 (1999)). GFP, a commonly used non-antibiotic selective marker, may cause cell death in certain cell lines (Hanazono et al., Hum Gene Ther. 8(11):1313-1319 (1997)). Thus, the cells and cell lines of this invention allow screening assays that are free from any artifact caused by selective drugs or markers. In some preferred embodiments, the cells and cell lines of this invention are not cultured with selective drugs such as antibiotics before or after cell sorting, so that cells and cell lines with desired properties are isolated by sorting, even when not beginning with an enriched cell population.

In another aspect, the invention provides methods of using the cells and cell lines of the invention. The cells and cell lines of the invention may be used in any application for which functional GC-C is needed. The cells and cell lines may be used, for example, but not limited to, in an in vitro cell-based assay or an in vivo assay where the cells are implanted in an animal (e.g., a non-human mammal) to, e.g., screen for GC-C modulators; produce protein for crystallography and binding studies; and investigate compound selectivity and dosing, receptor/compound binding kinetic and stability, and effects of receptor expression on cellular physiology (e.g., electrophysiology, protein trafficking, protein folding, and protein regulation). The cells and cell lines of the invention also can be used in knock down studies.

The present cells and cell lines may be used to identify the roles of different forms of GC-C in different GC-C pathologies by correlating the identity of in vivo forms of GC-C with the identify of known forms of GC-C based on their response to various modulators. This allows selection of disease- or tissue-specific GC-C modulators for highly targeted treatment of such GC-C-related pathologies.

Modulators include any substance or compound that alters an activity of GC-C. The modulator can be a GC-C agonist (potentiator or activator) or antagonist (inhibitor or blocker), including partial agonists or antagonists, selective agonists or antagonists and inverse agonists, and can be an allosteric modulator. A substance or compound is a modulator even if its modulating activity changes under different conditions or concentrations or with respect to different forms of GC-C. In other aspects, a modulator may change the ability of another modulator to affect the function of GC-C. For example, a modulator of a form of GC-C that is not activated by guanylin may render that form of GC-C susceptible to activation by guanylin.

To identify a GC-C modulator, one can expose a novel cell or cell line of the invention to a test compound under conditions in which GC-C would be expected to be functional and then detect a statistically significant change (e.g., p<0.05) in GC-C activity compared to a suitable control, e.g., cells that are not exposed to the test compound. Positive and/or negative controls using known agonists or antagonists and/or cells expressing different forms of GC-C may also be used. In some embodiments, the GC-C activity to be detected and/or measured is guanylyl cyclase activity, cGMP levels, stimulation or reduction of water or chloride secretion or regulation of mucosal and/or epithelial fluid absorption or secretion. One of ordinary skill in the art would understand that various assay parameters may be optimized, e.g., signal to noise ratio.

In some embodiments, one or more cells or cell lines of the invention are exposed to a plurality of test compounds, for example, a library of test compounds. A library of test compounds can be screened using the cell lines of the invention to identify one or more modulators. The test compounds can be chemical moieties including small molecules, polypeptides, peptides, peptide mimetics, antibodies or antigen-binding portions thereof. In the case of antibodies, they may be non-human antibodies, chimeric antibodies, humanized antibodies, or fully human antibodies. The antibodies may be intact antibodies comprising a full complement of heavy and light chains or antigen-binding portions of any antibody, including antibody fragments (such as Fab and Fab′, F(ab′)₂, Fd, Fv, dAb and the like), single chain antibodies (scFv), single domain antibodies, all or an antigen-binding portion of a heavy chain or light chain variable region.

In some embodiments, prior to exposure to a test compound, the cells or cell lines of the invention may be modified by pretreatment with, for example, enzymes, including mammalian or other animal enzymes, plant enzymes, bacterial enzymes, enzymes from lysed cells, protein modifying enzymes, lipid modifying enzymes, and enzymes in the oral cavity, gastrointestinal tract, stomach or saliva. Such enzymes can include, for example, kinases, proteases, phosphatases, glycosidases, oxidoreductases, transferases, hydrolases, lyases, isomerases, ligases and the like. Alternatively, the cells and cell lines may be exposed to the test compound first followed by treatment to identify compounds that alter the modification of the GC-C by the treatment.

In some embodiments, large compound collections are tested for GC-C receptor modulating activity in a cell-based, functional, high-throughput screen (HTS), e.g., using a 96 well, 384 well, 1536 well or higher format. In some embodiments, a test compound or multiple test compounds including a library of test compounds may be screened using more than one cell or cell line of the invention. In the case of a cell or cell line of the invention that expresses a human GC-C, one can expose the cells to a test compound to identify a compound that modulates GC-C activity (either increasing or decreasing) for use in the treatment of disease or condition characterized by undesired GC-C activity, or the decrease or absence of desired GC-C activity.

These and other embodiments of the invention may be further illustrated in the following non-limiting Examples.

### EXAMPLES

### Example 1

### Generating a Stable GC-C-Expressing Cell Line

293T cells were transfected with a plasmid encoding the human GC-C gene (SEQ ID NO: 2) using standard techniques. (Examples of reagents that may be used to introduce nucleic acids into host cells include, but are not limited to, LIPOFECTAMINE™, LIPOFECTAMINE™ 2000, OLIGOFECTAMINE™, TFX™ reagents, FUGENE® 6, DOTAP/DOPE, Metafectine or FECTURIN™.)

Although drug selection is optional in the methods of this invention, we included one drug resistance marker in the plasmid encoding the human GC-C gene. The GC-C sequence was under the control of the CMV promoter. An untranslated sequence encoding a tag for detection by a signaling probe was also present along with a sequence encoding a drug resistance marker. The target sequence utilized was Target Sequence 1 (SEQ ID NO: 1). In this example, the GC-C gene-containing vector contained Target Sequence 1.

Transfected cells were grown for 2 days in Dulbecco's Modified Eagles medium (DMEM)-FBS, followed by 10 days in 500 μg/ml hygromycin-containing DMEM-FBS, then in DMEM-FBS for the remainder of the time, totaling between 4 and 5 weeks (depending on which independent isolation) in DMEM/10% FBS, prior to the addition of the signaling probe.

Following enrichment on antibiotic, cells were passaged 8-10 times in the absence of antibiotic selection to allow time for expression that is not stable over the selected period of time to subside.

Cells were harvested and transfected with Signaling Probe 1 (SEQ ID NO: 11) using standard techniques. (Examples of reagents that may be used to introduce nucleic acids into host cells include, but are not limited to, LIPOFECTAMINE™, LIPOFECTAMINE™ 2000, OLIGOFECTAMINE™, TFX™ reagents, FUGENE® 6, DOTAP/DOPE, Metafectine or FECTURIN™.) The cells were then dissociated and collected for analysis and sorted using a fluorescence activated cell sorter.

Target Sequence 1 detected by Signaling probe 1
(SEQ ID NO: 1)
5′-GTTCTTAAGGCACAGGAACTGGGAC-3′
Signaling probe 1
(Supplied as 100 μM stock)
(SEQ ID NO: 11)
5′ - Cy5 GCCAGTCCCAGTTCCTGTGCCTTAAGAACCTCGC BHQ2 -3′

In addition, a similar probe using a QUASAR® Dye (BioSearch) with spectral properties similar to Cy5 was used in certain experiments. In some experiments, 5-MedC and 2-amino dA mixmers were used rather than DNA probes.

The cells were dissociated and collected for analysis and sorting using a fluorescence activated cell sorter. Standard analytical methods were used to gate cells fluorescing above background and to isolate individual cells falling within the gate into bar-coded 96-well plates. The following gating hierarchy was used: coincidence gate→singlets gate→live gate→Sort gate in plot FAM vs. Cy5: 0.3% of live cells

The above steps were repeated to obtain a greater number of cells. Two rounds of all the above steps were performed. In addition, the cell passaging, exposure to the signaling probe and isolation of positive cells by the fluorescence activated cell sorter sequence of steps was performed a total of two times for one of the independent transfection rounds.

The plates were transferred to a MICROLAB STAR™ (Hamilton Robotics). Cells were incubated for 9 days in 100 μl of 1:1 mix of fresh complete growth medium and 2-day-conditioned growth medium, supplemented with 100 U penicillin and 0.1 mg/ml streptomycin, dispersed by trypsinization twice to minimize clumps and transferred to new 96-well plates. Plates were imaged to determine confluency of wells (Genetix). Each plate was focused for reliable image acquisition across the plate. Reported confluencies of greater than 70% were not relied upon. Confluency measurements were obtained on 3 consecutive days and used to calculate growth rates.

Cells were binned (independently grouped and plated as a cohort) according to growth rate 3 days following the dispersal step. Each of the 4 growth bins was separated into individual 96-well plates; some growth bins resulted in more than one 96-well plate. Bins were calculated by considering the spread of growth rates and bracketing a range covering a high percentage of the total number of populations of cells. Bins were calculated to capture 12-hour differences in growth rate.

Cells can have doubling times from less than 1 day to more than 2 weeks. In order to process the most diverse clones that at the same time can be reasonably binned according to growth rate, it is preferable to use 3-9 bins with a 0.25 to 0.7 day doubling time per bin. One skilled in the art will appreciate that the tightness of the bins and number of bins can be adjusted for the particular situation and that the tightness and number of bins can be further adjusted if cells were synchronized for their cell cycle.

The plates were incubated under standardized and fixed conditions (DMEM/FBS, 37° C., 5% CO₂) without antibiotics. The plates of cells were split to produce 5 sets of 96-well plates (3 sets for freezing, 1 for assay and 1 for passage). Distinct and independent tissue culture reagents, incubators, personnel and carbon dioxide sources were used downstream in the workflow for each of the sets of plates. Quality control steps were taken to ensure the proper production and quality of all tissue culture reagents: each component added to each bottle of media prepared for use was added by one designated person in one designated hood with only that reagent in the hood while a second designated person monitors to avoid mistakes. Conditions for liquid handling were set to eliminate cross contamination across wells. Fresh tips were used for all steps, or stringent tip washing protocols were used. Liquid handling conditions were set for accurate volume transfer, efficient cell manipulation, washing cycles, pipetting speeds and locations, number of pipetting cycles for cell dispersal, and relative position of tip to plate.

One set of plates was frozen at −70 to −80° C. Plates in the set were first allowed to attain confluencies of 70 to 100%. Medium was aspirated and 90% FBS and 10% DMSO was added. The plates were individually sealed with Parafilm, surrounded by 1 to 5 cm of foam and placed into a freezer.

The remaining two sets of plates were maintained under standardized and fixed conditions as described above. All cell splitting was performed using automated liquid handling steps, including media removal, cell washing, trypsin addition and incubation, quenching and cell dispersal steps.

The consistency and standardization of cell and culture conditions for all populations of cells was controlled. Differences across plates due to slight differences in growth rates were controlled by normalization of cell numbers across plates and occurred 3 passages after the rearray. Populations of cells that are outliers were detected and eliminated.

The cells were maintained for 3 to 6 weeks to allow for their in vitro evolution under these conditions. During this time, we observed size, morphology, tendency towards microconfluency, fragility, response to trypsinization and average circularity post-trypsinization, or other aspects of cell maintenance such as adherence to culture plate surfaces and resistance to blow-off upon fluid addition.

Populations of cells were tested using functional criteria. The Direct Cyclic GMP Enzyme Immunoassay Kit (Cat. 900-014; AssayDesigns, Inc.) was used according to manufacturer's instructions: (http://www.assaydesigns.com/objects/catalog//product/extras/900-014.pdf). Cells were tested at 4 different densities in 96- or 384-well plates and responses were analyzed. The following conditions were used for the GC-C-expressing cell lines of the invention:

Clone screening: 1:2 and 1:3 splits of confluent 96-well plates 48 hour prior to assay, 30 minutes guanylin treatment.

Dose-response studies: densities of 20,000, 40,000, 60,000, 80,000, 120,000 and 160,000 per well, 30 minutes guanylin treatment (see Example 2).

Z′ studies: densities of 160,000 and 200,000 per well were used, 30 minutes guanylin treatment (see Example 3).

The functional responses from experiments performed at low and higher passage numbers were compared to identify cells with the most consistent responses over defined periods of time, ranging from 4 to 10 weeks. Other characteristics of the cells that changed over time were also noted.

Populations of cells meeting functional and other criteria were further evaluated to determine those most amenable to production of viable, stable and functional cell lines. Selected populations of cells were expanded in larger tissue culture vessels, and the characterization steps described above were continued or repeated under these conditions. At this point, additional standardization steps, such as different cell densities; time of plating, length of cell culture passage; cell culture dishes format and coating; fluidics optimization, including speed and shear force; time of passage; and washing steps, were introduced for consistent and reliable passages. Also, viability of cells at each passage was determined. Manual intervention was increased, and cells were more closely observed and monitored. This information was used to help identify and select final cell lines that retain the desired properties. Final cell lines and back-up cell lines (20 clones total) were selected that showed appropriate adherence/stickiness and growth rate and even plating (lack of microconfluency) when produced following this process and under these conditions.

The initial frozen stock of 3 vials per each of the selected 20 clones was generated by expanding the non-frozen populations from the re-arrayed 96-well plates via 24-well, 6-well and 10 cm dishes in DMEM/10% FBS/HEPES/L-Glu. The low passage frozen stocks corresponding to the cell lines were thawed at 37° C., washed two times with DMEM containing FBS and incubated in the same manner. The cells were then expanded for a period of 2 to 4 weeks. Two final clones were selected.

One vial from one clone of the initial freeze was thawed and expanded in culture. The resulting cells were tested to confirm that they met the same characteristics for which they were originally selected. Cell banks for each cell line consisting of 20 to over 100 vials may be established.

The following step can also be conducted to confirm that the cell lines are viable, stable and functional: At least one vial from the cell bank is thawed and expanded in culture; the resulting cells are tested to determine if they meet the same characteristics for which they were originally selected.

### Example 2

### Characterizing the Cell Lines for Native GC-C Function

A competitive ELISA for detection of cGMP was used to characterize native GC-C function in the produced GC-C-expressing cell line. Cells expressing GC-C were maintained under standard cell culture conditions in DMEM supplemented with 10% fetal bovine serum, glutamine and HEPES and grown in T175 cm flasks. For the ELISA, the cells were plated into coated 96-well plates (poly-D-lysine).

### Cell Treatment and Cell Lysis Protocol

Cells were washed twice with serum-free medium and incubated with 1 mM IBMX for 30 minutes. Desired activators (i.e., guanylin, 0.001-40 μM) were then added to the cells and incubated for 30-40 minutes. Supernatant was removed, and the cells were washed with TBS buffer. The cells were lysed with 0.1 N HCl. This was followed by lysis with 0.1N HCl and a freeze/thaw cycle at −20° C./room temperature. Defrosted lysates (samples were spun in Eppendorf tubes at 10,000 rpm) were centrifuged to pellet cell debris. The cleared supernatant lysate was then transferred to ELISA plates.

### ELISA Protocol

All of the following steps were performed at room temperature, unless otherwise indicated. ELISA plates were coated with anti-IgG antibodies in coating buffer (Na-carbonate/bi-carbonate buffer, 0.1 M final, pH 9.6) overnight at 4° C. Plates were then washed with wash buffer (TBS-Tween 20, 0.05%), followed by blocking reagent addition. Incubation for 1 hour with blocking reagent at 37° C. was followed by a wash of the plates with wash buffer. A rabbit anti-cGMP polyclonal antibody (Chemicon) was then added, followed by incubation for 1 hour and a subsequent wash with wash buffer. Cell lysate was then added, and incubated for 1 hour before the subsequent addition of a cGMP-biotin conjugate (1 and 10 nM of 8-Biotin-AET-cGMP (Biolog)). Plates were incubated for 2 hours and then washed with wash buffer. Streptavidin-alkaline phosphate was then added and incubated for 1 hour, then washed with wash buffer. Plates were incubated for at least 1 hour (preferably 2-5 hours) with PNPP substrate (Sigma). The absorbance was then read at 405 nm on a SAFIRE²™ plate reader (Tecan).

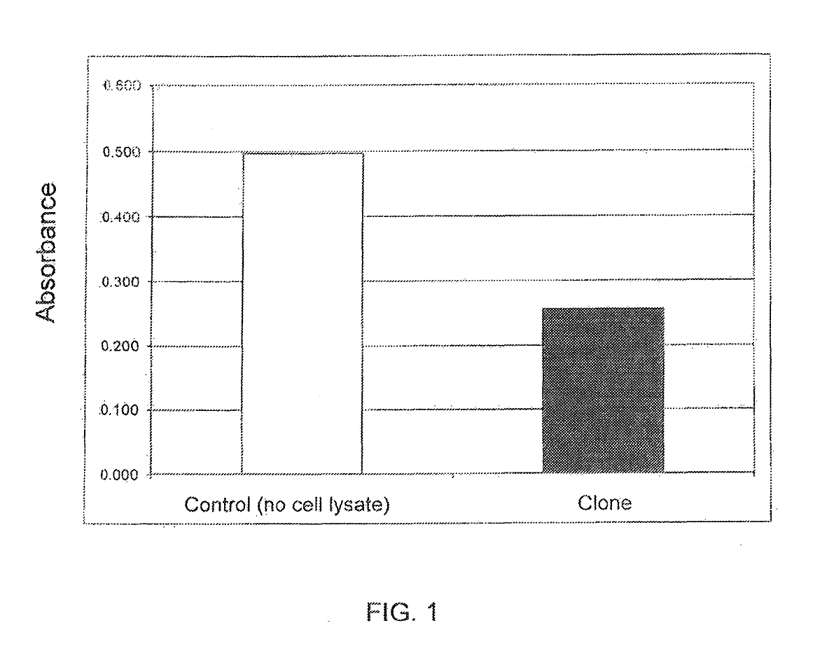

Representative data from the competitive ELISA is presented in FIG. 1. Maximum absorbance was seen when no cell lysate was used in the ELISA (Control). Reduction in absorbance (corresponding to increased cGMP levels) was observed with cell lysate from the produced GC-C-expressing cell line treated with 100 nM guanylin (Clone).

The cGMP level in the produced GC-C-expressing cell line treated with 100 nM guanylin was also compared to that of parental cell line control samples not expressing GC-C (not shown) using the Direct Cyclic GMP Enzyme Immunoassay Kit (Cat. 900-014; AssayDesigns, Inc.). The GC-C-expressing cell line showed a greater reduction in absorbance (corresponding to increased cGMP levels) than parental cells treated and untreated with guanylin.

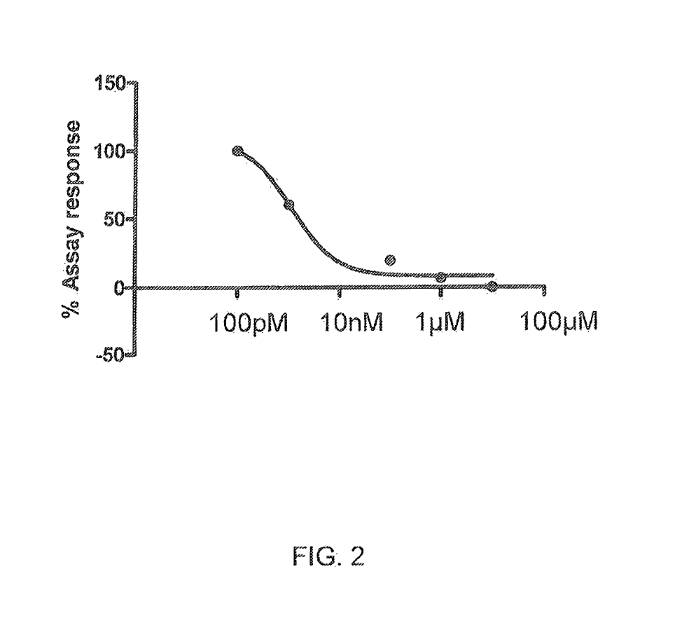

For guanylin dose-response experiments, cells of the produced GC-C-expressing cell line, plated at densities of 20,000, 40,000, 60,000, 80,000, 120,000 and 160,000 cells/well in a 96-well plate, were challenged with increasing concentration of guanylin for 30 minutes (see FIG. 2). The cellular response (i.e., absorbance) as a function of changes in cGMP levels (as measured using the Direct Cyclic GMP Enzyme Immunoassay Kit (Cat. 900-014; AssayDesigns, Inc.) was detected using a SAFIRE²™ plate reader (Tecan). Data were then plotted as a function of guanylin concentration and analyzed using non-linear regression analysis using GraphPad Prism 5.0 software, resulting in an EC₅₀ value of 1.1 nM. (FIG. 2). The produced GC-C-expressing cell line shows a higher level of cGMP (6 pmol/ml) when treated with low concentrations of guanylin in comparison to that previously reported in other cell lines (3.5 pmol/ml) (Forte et al., Endocr. 140(4):1800-1806 (1999)), indicating the potency of the clone.

### Example 3

### Generation of GC-C-Expressing Cell Line Z′ Value

Z′ for the produced GC-C-expressing cell line was calculated using a direct competitive ELISA assay. The ELISA was performed using the Direct Cyclic GMP Enzyme Immunoassay Kit (Cat. 900-014; AssayDesigns, Inc.). Specifically, for the Z′ assay, 24 positive control wells in a 96-well assay plate (plated at a density of 160,000 or 200,000 cells/well) were challenged with a GC-C activating cocktail of 40 μM guanylin and IBMX in DMEM media for 30 minutes. Considering the volume and surface area of the 96-well assay plate, this amount of guanylin created a concentration comparable to the 10 μM used by Forte et al. (1999) Endocr. 140(4), 1800-1806. An equal number of wells containing clonal cells in DMEM/IMBX were challenged with vehicle alone (in the absence of activator). Absorbance (corresponding to cGMP levels) in the two conditions was monitored using a SAFIRE²™ plate reader (Tecan). Mean and standard deviations in the two conditions were calculated and Z′ was computed using the method of Zhang et al., J Biomol Screen, 4(2):67-73 (1999)). The Z′ value of the produced GC-C-expressing cell line was determined to be 0.72.

### Example 4

### Short-Circuit Current Measurements

Ussing chamber experiments are performed 7-14 days after plating GC-C-expressing cells (primary or immortalized epithelial cells, for example, lung, intestinal, mammary, uterine, or renal) on culture inserts (Snapwell, Corning Life Sciences). Cells on culture inserts are rinsed, mounted in an Ussing type apparatus (EasyMount Chamber System, Physiologic Instruments) and bathed with continuously gassed Ringer solution (5% CO₂ in O₂, pH 7.4) maintained at 37° C. containing (in mM) 120NaCl, 25NaHCO₃, 3.3KH₂PO₄, 0.8K₇HPO₄, 1.2CaCl₂, 1.2MgCl₂, and 10 glucose. The hemichambers are connected to a multichannel voltage and current clamp (VCC-MC8, Physiologic Instruments). Electrodes [agar bridged (4% in 1 M KCl) Ag-AgCl] are used, and the inserts are voltage clamped to 0 mV. Transepithelial current, voltage and resistance are measured every 10 seconds for the duration of the experiment. Membranes with a resistance of <200 mOhms are discarded. This secondary assay can provide confirmation that in the appropriate cell type (i.e., cell that form tight junctions) the introduced GC-C is altering CFTR activity and modulating a transepithelial current.

1. A cell or cell line engineered to stably express a functional guanylate cyclase C (GC-C), wherein the GC-C optionally is expressed from an introduced nucleic acid encoding it.

2-3. (canceled)

4. The cell or cell line of claim 1, wherein the GC-C is expressed from an endogenous nucleic acid engineered by gene activation.

5. The cell or cell line of claim 1, which
a) is eukaryotic;
b) is mammalian;
c) does not express endogenous GC-C prior to engineering; or
d) is any combination of a), b) and c).

6. (canceled)

7. The cell or cell line of claim 1, wherein the GC-C is human.

8. The cell or cell line of claim 1, which produces a Z′ factor of at least 0.4 in an assay.

9. The cell or cell line of claim 8, wherein the cell or cell line is maintained without selective pressure, and wherein the GC-C optionally comprises a polypeptide tag.

10-12. (canceled)

13. The cell or cell line of claim 12, wherein said cell or cell line expresses the GC-C in the absence of selective pressure for at least 15 days, at least 30 days, at least 45 days, at least 60 days, at least 75 days, at least 100 days, at least 120 days, or at least 150 days.

14. (canceled)

15. The cell or cell line of claim 1, which is suitable for use in a high throughput screening assay, wherein the GC-C produces a detectable signal-to-noise ratio greater than 1.

16-18. (canceled)

19. The cell or cell line of claim 1, wherein the GC-C is selected from the group consisting of:
a) a GC-C polypeptide comprising the amino acid sequence set forth in SEQ ID NO: 3;
b) a GC-C polypeptide comprising an amino acid sequence that is at least 95% identical to the amino acid sequence of SEQ ID NO: 3;
c) a GC-C polypeptide encoded by a nucleic acid that hybridizes under stringent condition to SEQ ID NO: 2; and
d) a GC-C polypeptide that is encoded by an allelic variant of SEQ ID NO: 2.

20. The cell or cell line of claim 1, wherein the GC-C is encoded by a nucleic acid selected from the group consisting of:
a) a nucleic acid comprising the sequence set forth in SEQ ID NO: 2;
b) a nucleic acid that hybridizes to a nucleic acid comprising the nucleotide sequence of SEQ ID NO: 2 under stringent conditions;
c) a nucleic acid that encodes a polypeptide comprising the amino acid sequence of SEQ ID NO: 3;
d) a nucleic acid comprising a nucleotide sequence that is at least 95% identical to SEQ ID NO: 2; and e) a nucleic acid that is an allelic variant of SEQ ID NO: 2.

21. A collection of the cell or cell line of claim 1, wherein the cells or cell lines in the collection express different forms or mutants of GC-C.

22. (canceled)

23. A collection of the cell or cell line of claim 1, wherein at least one cell or cell line expresses an introduced receptor other than GC-C.

24. (canceled)

25. The collection of claim 21 or 23, wherein the cells or cell lines are matched to share the same physiological property to allow parallel processing.

26-29. (canceled)

30. A method for producing the cell or cell line of claim 1 or the collection of claim 21 or 23 comprising the steps of:
a) introducing into host cells a nucleic acid encoding GC-C or one or more nucleic acid sequences that activate expression of endogenous GC-C;
b) introducing into the host cells a molecular beacon that detects the expression of GC-C or activated GC-C in the host cells; and
c) isolating a cell that expresses GC-C or activated GC-C, wherein said isolating optionally utilizes a fluorescence activated cell sorter.

31-33. (canceled)

34. The method of claim 30, wherein the GC-C comprises an amino acid sequence that is selected from the group consisting of:
a) the amino acid sequence set forth in SEQ ID NO: 3, and;
b) the amino acid sequence encoded by the nucleic acid comprising SEQ ID NO: 2.

35-37. (canceled)

38. A method for identifying a modulator of a GC-C function, comprising the step of exposing the cell or cell line of claim 1 to a test compound and detecting a change in a GC-C function in a cell compared to a cell not contacted with the test compound, wherein a change in said function indicates that the test compound is a GC-C modulator.

39-40. (canceled)

41. The method of claim 38, wherein the GC-C is human GC-C.

42. The method of claim 38, wherein the test compound is a small molecule, a chemical moiety, a polypeptide, an antibody, or an antigen binding portion of an antibody.

43-44. (canceled)

45. A method for identifying a modulator of any introduced protein, comprising the step of exposing the collection of any one of claims 21, 23 or 25 to a test compound and detecting a change in the function of the introduced protein in a cell compared to a cell not contacted with the test compound, wherein a change in said function indicates that the test compound is a modulator of the introduced protein.

46. The method of claim 45, wherein the modulator affects the function of all the introduced proteins in the collection.

47-53. (canceled)

54. A cell engineered to stably express a GC-C at a consistent level over time, the cell made by a method comprising the steps of:
a) providing a plurality of cells that express mRNA encoding the GC-C;
b) dispersing the cells individually into individual culture vessels, thereby providing a plurality of separate cell cultures;
c) culturing the cells under a set of desired culture conditions using automated cell culture methods characterized in that the conditions are substantially identical for each of the separate cell cultures, during which culturing the number of cells per separate cell culture is normalized, and wherein the separate cultures are passaged on the same schedule;
d) assaying the separate cell cultures to measure expression of the GC-C at least twice; and
e) identifying a separate cell culture that expresses the GC-C at a consistent level in both assays, thereby obtaining said cell.
